(19) United States
(12) Patent Application Publication
(10) Pub. No.: US 2023/0251260 A1
(43) Pub. Date: Aug. 10, 2023

(54) METHODS OF DETERMINING CANCER THERAPY

(71) Applicant: YISSUM RESEARCH DEVELOPMENT COMPANY OF THE HEBREW UNIVERSITY OF JERUSALEM LTD., Jerusalem (IL)

(72) Inventor: Nataly Kravchenko-Balasha, Kfar Uriya (IL)

(21) Appl. No.: 18/004,673

(22) PCT Filed: Jul. 8, 2021
(86) PCT No.: PCT/IB2021/056136

§ 371 Date: Jan. 8, 2023

Related U.S. Application Data
(60) Provisional application No. 63/049,664, filed on Jul. 9, 2020.

(51) Int. Cl. G01N 33/574; G16B 20/00; G16B 25/10

(52) U.S. Cl. CPC G01N 33/574; G16B 20/00; G16B 25/10; G01N 2570/00

(57) ABSTRACT

Methods of determining a therapy for a solid cancer comprising thermodynamic-based analysis of single-cell proteomic data from tumor-derived cells are provided. Methods for determining a combination therapy comprising thermodynamic-based analysis of single cell proteomic data from tumor-derived cells that have received a first therapy are also provided. Methods of treating a subject suffering from triple-negative breast cancer, comprising administering radiotherapy, anti-Her2 therapy and anti-cMet therapy are also provided.

### CROSS-REFERENCE TO RELATED APPLICATIONS

This application claims the benefit of priority from U.S. Provisional Patent Application No. 63/049,664 filed 9 Jul. 2020, and titled “METHODS OF DETERMINING CANCER THERAPY”, the contents of which are incorporated herein by reference in their entirety.

### FIELD OF INVENTION

The present invention is in the field of cancer therapy.

### BACKGROUND OF THE INVENTION

Cancer is a complex disease, characterized by a malfunctioning of signaling networks. Aberrant signaling events play key roles in the maintenance and progression of tumors. This understanding has spurred the development of targeted therapies, specifically aimed at proteins that transduce signals through the defective pathways. However, though targeted anti-cancer therapy initially showed considerable promise, it soon became clear that single targeted agents seldom suffice to induce complete tumor remission. The molecular variability among different tumors, referred to as inter-tumor heterogeneity, greatly complicates the prediction of the tumor's response to the treatment, and therefore the designation of the appropriate therapy.

This difficulty is exacerbated by intra-tumor heterogeneity, the variability among different cellular populations of a single tumor. Even when a first line therapy is initially effective, relapse can occur due to overgrowth of a small population of tumor cells that are not affected by the first therapy. Further, when tumor-wide expression is examined the key regulators in small populations can be missed due to the noise produced from the high variability between cells.

A plethora of analytical methods that address the complex nature of protein networks have been developed: Bayesian methods, based on elucidating the relationships between a few genes at a time; reverse-engineering algorithms, based on chemical kinetic-like differential equations; and multivariate statistical methods that include clustering methods, principal component analysis, singular value decomposition and meta-analysis. These methods have significantly progressed the fields of computational analysis and cancer research. However, the majority of aggressive tumors still do not respond well to therapy.

Surprisal analysis (SA) is an information theoretic, thermodynamic-like technique. It examines protein-protein correlations and, based on information theoretic and thermodynamic-like considerations, identifies the constraints that operate in the studied system as well as the proteins that were affected by each constraint. Previously, surprisal analysis of cell-cell signaling in brain tumors provided a prediction about cellular spatial distributions and the direction of cell-cell movement (Kravchenko-Balasha, N., et al., Glioblastoma cellular architectures are predicted through the characterization of two-cell interactions. Proc Natl Acad Sci USA 111, 6521-6526 (2014) and Kravchenko-Balasha N, et al, Intercellular Signaling Through Secreted Proteins Induces Free Energy Gradient-directed Cell Movement. Proc Natl Acad Sci USA 113(20), 5520-5 (2016)). SA has also been implemented to tumor epithelial-to-mesenchymal transitions (EMT) (Poovathingal et al., Critical points in tumorigenesis: a carcinogen initiated phase transition analyzed via single cell proteomics. Small 12(11): 1425-1431 (2016)) and to large-scale whole proteomic datasets obtained from multiple cancer types (Flashner-Abramson, E., et al., Decoding cancer heterogeneity: studying patient-specific signaling signatures towards personalized cancer therapy. Theranostics 9, 5149-5165 (2019)). SA successfully predicted efficient patient-specific targeted combination therapies; however, these methods did not address the vast intratumor cellular heterogeneity that exists in many cancers, and the dynamic changes that occur in a tumor after exposure to therapy.

Further, certain types of cancer are even more confounding. This includes cancers such as triple negative breast cancer (TNBC), which is an aggressive type of breast cancer that lacks known biomarkers for targeted therapy. Instead, the common treatment is chemotherapy and/or radiation treatment (RT), which also often leads to relapse. Recent studies have shown that radiation, while effectively killing cancer cells, also promotes anti-apoptotic and pro-proliferative responses that often result in tumor regrowth. This notion gave rise to numerous studies attempting to characterize tumor molecular phenotypes occurring in response to radiation, in order to develop new strategies to enhance the response of cancer to radiotherapy. These studies, however, must deal with not only inter-patient heterogeneity, but significant variability between tumor cells within the tumor. Not a single targeted therapy has been approved for the treatment of TNBC, and thus chemotherapy (CT) and radiation therapy (RT) remain the standard treatments over the past 20 years. Methods of determining effective targeted treatment in various cancers, as well as specifically finding second line cancer treatments to combine with the first line treatment and thus avoid relapse are greatly needed. Further, methods that examine the intra-tumor heterogeneity of cellular populations, rather than looking at the entire tumor proteome are also greatly needed.

### SUMMARY OF THE INVENTION

The present invention provides methods of determining a therapy for a solid cancer comprising thermodynamic-based analysis of single-cell proteomic data from tumor-derived cells. Methods for determining a combination therapy comprising thermodynamic-based analysis of single cell proteomic data from tumor-derived cells that have received a first therapy are also provided, as are methods of treating a subject suffering from triple-negative breast cancer, comprising administering radiotherapy, anti-Her2 therapy and anti-cMet therapy are also provided.

According to a first aspect, there is provided a method of selecting a therapy for a solid cancer, the method comprising:
a. receiving a single-cell proteomic analysis of a population of cells derived from the solid cancer;
b. calculating for the population of cells at least two unbalanced processes active in at least one cell of the population of cells, wherein the calculating comprises performing a deterministic thermodynamic-based analysis on the proteomic analysis, thereby producing a list of calculated unbalanced processes;
c. generating for a plurality of cells of the population of cells a cell-specific signature (CSS) comprising all unbalanced processes from the list of calculated unbalanced processes active in each cell of the plurality of cells;
d. assigning all cells with the same CSS to a cellular population; and
e. selecting a therapy that targets a CSS of at least one cellular population;
thereby selecting a therapy.

According to another aspect, there is provided a computer program product for determining a therapy for a solid cancer, comprising a non-transitory computer-readable storage medium having program code embodied thereon, the program code executable by at least one hardware processor to
f. receive a single-cell proteomic analysis of a population of cells derived from a solid cancer;
g. determine for the population of cells at least two unbalanced processes active in at least one cell of the population of cells, wherein the determining comprises performing a deterministic thermodynamic-based analysis on the proteomic analysis, thereby producing a list of determined unbalanced processes;
h. generate for a plurality of cells of the population of cells a cell-specific signature (CSS) comprising all unbalanced processes from the list of determined unbalanced processes active in each cell of the plurality of cells;
i. assign all cells with the same CSS to a cellular population; and
j. select and output a therapy that targets a CSS of at least one cellular population.

According to some embodiments, the receiving is receiving a single-cell proteomic analysis of a population of cells at a given timepoint.

According to some embodiments, the method of the invention further comprises obtaining a sample comprising cells from the solid cancer and subjecting the cells to a single-cell proteomic analysis.

According to some embodiments, the sample is digested into a single-cell suspension before proteomic analysis of single cells.

According to some embodiments, the proteomic analysis comprises single-cell FACS analysis.

According to some embodiments, the FACS analysis is surface protein analysis.

According to some embodiments, the proteomic analysis is analysis of a plurality of oncogenic proteins.

According to some embodiments, activity of the oncogenic proteins is characteristic of tumors of the cancer.

According to some embodiments, the proteomic analysis analyzes at least 10 proteins.

According to some embodiments, the solid cancer is a tumor of a subject, and the method is a method of selecting a subject-specific therapy.

According to some embodiments, the solid cancer is a tumor of a model organism baring a human tumor or a model for a human tumor or is a human tumor cell line and the method is a method of selecting a general anti-tumor therapy.

According to some embodiments, the deterministic thermodynamic-based analysis is surprisal analysis.

According to some embodiments, the calculating at least two unbalanced processes active in at least one cell of the population of cells is calculating all unbalanced processes active in at least one cell of the population of cells based on the thermodynamic-based analysis.

According to some embodiments, the CSS comprises all significantly unbalanced processes from the list of calculated unbalanced process active in cells of the population.

According to some embodiments, a significantly unbalanced process comprises a significant amplitude.

According to some embodiments, the method of the invention further comprises administering to a subject suffering from the solid cancer the selected therapy.

According to some embodiments, the subject provided the cells derived from the solid cancer.

According to some embodiments, the population of cells has been contacted by a first therapy prior to the proteomic analysis and the method is a method of selecting a second therapy, or a combination therapy of the first therapy and a second therapy.

According to some embodiments, the solid cancer is from a subject and the subject has been administered the first therapy prior to derivation of the population of cells.

According to some embodiments, prior comprises a period of time sufficient for at least 80% regrowth of the tumor.

According to some embodiments, the period of time sufficient for regrowth is at least 6 days.

According to some embodiments, the method of the invention further comprises receiving a single-cell proteomic analysis of a population of cells derived from the solid cancer before treatment with the first therapy, and assigning all cells of a plurality of cells of the population of cells derived from the cancer before treatment to a cellular population, wherein the selecting a second therapy is selecting a second therapy that targets a CSS of at least one cellular population that increased in abundance following the first therapy.

According to some embodiments, the assigning comprises calculating for the population of cells derived before treatment at least two unbalanced processes active in at least one cell of the population of cells derived before treatment, wherein the calculating comprises performing a deterministic thermodynamic-based analysis on the proteomic analysis, thereby producing a list of calculated unbalanced processes; generating for the plurality of cells a CSS comprising all unbalanced processes from the list of calculated unbalanced processes active in each cell of the plurality of cells of the population of cells derived from the cancer before treatment, and assigning all cells with the same CSS to a cellular population.

According to some embodiments, the method of the invention comprises calculating the percent of all cells of the population before treatment that are in each cellular population, and calculating the percent of all cells of the population following treatment that are in each cellular population, and wherein an increase in abundance of a cellular population is an increase in the percent the population is of the total.

According to some embodiments, the method of the invention comprises selecting therapies that target CSS of a plurality of cellular populations that increase in abundance following the first therapy.

According to some embodiments, the combination therapy is coadministration of the first and the second therapy, or pre-administration of the second therapy before administering the first therapy.

According to some embodiments, the first therapy is an untargeted cancer therapy.

According to some embodiments, the untargeted cancer therapy is selected from radiotherapy, immune cell transfer and chemotherapy.

According to some embodiments, the untargeted cancer therapy is radiotherapy.

According to some embodiments, the second therapy is a targeted therapy that targets a protein of an unbalanced process of the CSS.

According to some embodiments, the method of the invention further comprises administering to a subject suffering from the solid cancer the determined second therapy or combined therapy.

According to some embodiments, the subject provided the population of cells derived from the solid cancer.

According to some embodiments, the population of cells comprises at least 50,000 cells.

According to some embodiments, the method is a computer implemented method.

According to another aspect, there is provided a method of treating a subject suffering from triple-negative breast cancer, the method comprising administering to the subject radiotherapy, anti-Her2 therapy and anti-cMet therapy, thereby treating triple-negative breast cancer.

According to some embodiments, the anti-Her2 therapy and the anti-cMet therapy are administered concomitantly.

According to some embodiments, the anti-Her2 therapy and the anti-cMet therapy are administered before or concomitantly to the radiotherapy.

According to some embodiments, the method of the invention further comprises determining that the triple-negative breast cancer comprises minor cell populations with CSSs targetable by anti-Her2 therapy and anti-cMet therapy.

According to some embodiments, the anti-Her2 therapy is Herceptin.

According to some embodiments, the anti-cMet therapy is Crizotinib.

According to another aspect, there is provided anti-Her2 therapy and anti-cMet therapy for use in combination with radiotherapy for treating triple negative breast cancer in a subject in need thereof.

According to another aspect, there is provided a kit comprising an anti-Her2 therapy and an anti-cMet therapy and a label stating the anti-Her2 therapy and anti-cMet therapy are for use in combination with radiotherapy.

According to some embodiments, the kit of the invention is for use in treating triple negative breast cancer in a subject in need thereof.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Decoding intratumor heterogeneity into distinct subpopulations after radiation treatment can offer new targets for tumor-specific therapy. Phenotypic variations due to intratumor heterogeneity have been a critical challenge towards gaining the optimal therapy for each patient. Utilizing high throughput flow cytometry and single cell analysis, based on Surprisal analysis, there is identified a patient-specific structure of the tumor. Cellular subpopulations and altered processes are recognized in each subpopulation before and after radiotherapy. Accurate resolution and targeting of aggressive cellular subpopulations aim to prevent the expansion of resistant subpopulations and to sensitize the tumor to radiation treatment.

FIGS. 2A-F: Scheme of the algorithm of Surprisal analysis. (2A) Preparation of single cell suspension from different samples. After irradiation treatment, the tumor mass/cultured cells are mechanically disrupted into single cell suspension which is later labelled with fluorescently tagged antibodies and run on the cytometer. Using (30,000-50,000) cells per sample, surprisal analysis identifies distributions of the protein expression levels at the reference (balanced) state and deviations thereof (2B). (2C) Proteins that deviate in a similar manner from the references (e.g. both induced in a certain group of cells) are grouped into altered subnetworks, named “unbalanced processes”. (2D) Several unbalanced processes may be active in one cell. Thus, we calculate an amplitude of each process in each cell. Processes with significant amplitudes are assigned to each cell, thereby providing a cell specific signaling signature (CSSS) for each cell. Each CSSS is transformed into a cell specific barcode. (2E) Cells sharing the same barcode are organized into distinct subpopulations. (2F) Finally, a tumor-specific combination of targeted therapies is tailored to CSSS.

FIGS. 3A-P: Resolution of expanded subpopulations in 4T1 cellular population post irradiation. (3A) FACS expression levels of Her2 and cMet following RT (control, 5 Gy and 15 Gy) are shown. (3B) Raw FACS data of expression levels of 11 proteins, before and in response to low dose (5Gy) or high dose (15 Gy) irradiation are shown as one-dimensional boxplots. (3C-E) FACS experimental data plotted as correlation plots between Her2 and cMet (3C), Her2 and EGFR (3D), and MUC land cMet (3E). (3F) The 10 unbalanced subnetworks (processes) resolved in 4T1 are shown. Line connections were drawn using String database. (3G) Each cell was assigned a barcode representing CSSS. The group of cells harboring the same barcode was defined as a subpopulation. Most abundant (>1%) subpopulations are presented. (3H) Exemplary scatter plot representation of λ3(cell,t) values for process 3 in 4T1 irradiated cells. The amplitude of unbalanced process 3 is plotted in order to follow up with the information this process is providing. The proteins that are participating in this process are Her2 and to a lesser extent EGFR. Based on the calculation of G_{iα}λ_{α} (cell,t) product, the upregulated and downregulated protein expression levels are being defined. Due to this process 3, EGFR and Her2 are being altered in the same direction (EGFR is upregulated and Her2 is also upregulated or vice versa due to the process). The other 9 proteins used in the panel are not affected by this process. Similar plots are built for all lambda values. (3I) Sigmoid plotting of λ1(cell) values to identify the thresholds (limits) of significant values posing an altered process 1. Active processes in different cells were identified as follows: For every unbalanced process α, λ_{α} (cell) values were sorted according to their values, and only cells with significant λ_{α} (cell) values were considered to possess an unbalanced process α. This is exemplified for the process α=1 of 4T1 cells post to RT as shown in the figure. Shown are sorted values of λ₁ (cell), which represent an amplitude of the process α=1 in each cell. The black lines represent threshold values. In this example cells received the values λ_{α} (cell)>0.5 or λ_{α} (cell)<−0.5 (which form the top and bottom “tails” of the distribution) for the process #1, were considered to possess the unbalanced process α=1 and used in the python script in order to obtain the barcodes shown in FIG. 3G. These values were used to calculate further the products G_{iα}λ_{α}(cell,t) in order to build a functional subnetwork using STRING database, presented in FIG. 3F. (3J) Correlation plot between Her2 and EGFR expression levels includes the cells with significant λ₃ (cell) values. (3K) Schematic representation of the temporal behavior of abundant subpopulations is demonstrated. (3L) Based on the CSSSs the tumor was divided into distinct subpopulations. (3M) Table of G values for 4T1 irradiated cells. G_{iα} are weights of a protein i in the unbalanced processes (α=1, 2 . . . ). Significant G_{iα} values are labeled in blue and red colors. The sign of the G value indicates the correlation or anti-correlation between proteins in the same process as shown in the table. G_{Her2 1}=0.4 and G_{EGFR 1}=−0.9 indicating that in process #1 the two altered proteins are Her2 and EGFR which are anti-correlated due to the process. (3N) Very small subpopulations (<<1%), represented by barcodes b and f, expanded significantly following RT. (3O) Survival rates of 4T1 cells in response to Herceptin (H), Crizotinib (C), radiation therapy (RT), RT+C, RT+H and RT+H+C as detected by survival assays 6 days post RT. Drugs were added 3 days prior to RT and kept until the end of the experiment. SD are shown. (3P) Downstream signaling to Her2 and cMet, as represented by the key downstream proteins, is shown following different treatments. The predicted combination induced high levels of cleaved caspase-3 compare to radiation alone, RT+H and RT+C. Downregulation of pAKT, pERK and p-S6 was detected when H+C was applied prior to RT.

FIGS. 4A-E: Inhibition of the expanded subpopulations following RT sensitized the tumor response to RT. (4A) 6-7 week-old Balb/C female mice were subcutaneously injected with 4T1 cells. Mice were RT treated by on two alternate days (12Gy) with brachytherapy when tumor volumes reached (80-100) mm³. (4B) Tumor volumes in control group (red) and RT group (black) in response to RT. Day 1 is the 1st day after completing the 2nd irradiation. Brachytherapy applied on day −2 and 0. SD and p values of t-test are shown in the graph. (4C) Fold change in the abundancy of the subpopulations b and f as compare to untreated tumors. A significant expansion due RT in subpopulation b harboring Her2⁺/EGFR⁺ and subpopulation f harboring cMet⁺/MUC1⁺ is detected. The size of theses subpopulations did not change significantly after tumor regrowth (day 12). SD are shown. (4D) Mice were subcutaneously injected with 4T1 cells. Mice were treated by brachytherapy as in panel (4A). Herceptin (H), 5 mg/kg and Crizotinib (C), 25 mg/kg, were administrated IP 2 d/week and by gavage 5 d/week correspondingly on d-5 (2 days before RT) and until the end of the experiment (d12). St. errors and p values are shown. (4E) In-vivo fold change in the abundance of the subpopulations b and f shows the expansion of subpopulations b and f and the size reduction of these subpopulations when H and C were used as a predicted pretreatment along with RT. These results were consistent 6 days and 12 days after irradiation (RT).

FIGS. 5A-G: Inhibition of the expanded subpopulations sensitizes human TNBC and BR45 PDX to RT. (5A) Survival assays demonstrate that 6 days post RT, 30% of cells survived. 14 days post RT TNBC regrow to ˜80-90% confluency. (5B) Fold change in the abundance of the subpopulation b and f compare to untreated cells. These subpopulations either remained unchanged or expanded after tumors' regrowth. (5C) Survival rates of Br45, MD-468 and MD-231 cells in response to Herceptin (H)+Crizotinib (C), Herceptin (H)+Erlotinib (E), RT, RT+H, RT+C, RT+H+E and RT+H+C 6 days post RT. Drugs were added 2 days prior to RT and kept until the end of the experiment. For FIGS. 5A-C SD are shown. (5D) Downstream to Her2 and cMet signaling following different treatments. C+H combination induced higher levels of cleaved caspase-3, compare to irradiation alone and irradiation with either C or H alone. C+H combination applied prior to RT induced downregulation of pAKT, pERK and p-S6 levels (FIG. 3G). (5E) C+H sensitizes TNBC response to RT in BR45 PDX in-vivo. BR45 tissues were transplanted orthotopically to 60 (6-7 week-old) NSG female mice. Mice were treated by brachytherapy RT on two alternative days (d2 and d0) with 12Gy and 10Gy respectively. Drugs were administrated on d5 (2 days before RT) and until the end of the experiment (d12). For doses see SI methods. St. errors are shown. (5F) In-vivo fold change showing the expansion of subpopulations b and f and the reduction of these subpopulations when H and C were used. (5G) One-dimensional boxplots show the upregulation of Her2 (left) and cMet (right) after irradiation on two alternative days with (10 or 12) Gy respectively and the downregulation of these proteins when CSSS-predicted targeted therapy was applied.

### DETAILED DESCRIPTION OF THE INVENTION

The present invention, in some embodiments, provides methods of determining a therapy for a solid cancer. The present invention further concerns methods for determining a combination therapy for a solid cancer. Methods of treating a subject suffering from triple-negative breast cancer (TNBC) are also provided.

The invention is based on the surprising finding that an information theoretic technique, named surprisal analysis (SA), can be used to resolve solid tumor cellular subpopulations on the single cell level, evaluate their response to first line therapy, and design a therapeutic method to sensitize the tumor cells to that therapy. This has been exemplified in TNBC.

Tumors are considered to be homeostatically disturbed entities, which have deviated from their balanced state due to various constraints (e.g. mutational stress, application of drugs, etc.). Each constraint creates a deviation in the expression levels of a subset of proteins in the tumor. Thus, a constraint creates an unbalanced process in the tumor, consisting of the group of proteins that was altered by the constraint. SA examines protein-protein correlations and based on information theoretic and thermodynamic-like considerations, identifies the constraints that operate in the studied system as well as the proteins that were affected by each constraint.

Unlike previous studies, herein SA is utilized to study single cells. For each cell a cell-specific signaling signature—CSSS is identified, consisting of the complete set of unbalanced processes that emerged within the individual cell. An intra-tumor subpopulation is then defined to be a group of cells harboring the exact same CSSS. These cells are expected to respond similarly to treatment.

The final result of the analysis is a high-resolution intra-tumoral map of the different subpopulations within the tumor, and the CSSS that operates in every subpopulation. Importantly, even very small subpopulations (as low as 100 cells) can be captured using this technique. Such a robust and comprehensive map can be a guide to the proper determination of drug combinations that will effectively target dominant subpopulations, as well as small and persistent subpopulations within the tumor, and bring about a potent effect.

This finding was exemplified in a 4T1 murine model for stage IV triple-negative breast cancer (TNBC), as well as human TNBC and patient-derived xenograft models. It was shown that upon radiotherapy treatment in-vitro and in-vivo, all models demonstrate a significant expansion of two distinct cellular subpopulations: one with upregulated EGFR/Her2 and another with upregulated cMet/MUC1. Those subpopulations are hardly detectable in untreated tumors. It is hypothesized that poor response of TNBC to RT can be overcome by inhibiting the growth of those subpopulations. We validate our hypothesis by showing that RT-treated TNBC tumors that were simultaneously pretreated with anti-Her2 (Herceptin) and anti-cMet (Crizotinib) inhibitors do not relapse, in vitro and in vivo. Each targeted drug alone demonstrates a significantly smaller effect.

In summary, herein is provided a novel framework for the resolution of tumor-specific cellular heterogeneity at the single cell level. It is shown that accurate mapping of tumor cellular subpopulations within a cancer mass can provide guidance on mono-treatments as well as combination therapies.

By a first aspect, there is provided a method of identifying at least one subpopulation within a population of cells, the method comprising:
k. receiving a single-cell expression analysis of the population of cells;
l. calculating for the population of cells at least one unbalanced process active in at least one cell of the population of cells;
m. generating for a plurality of cells of the population of cells a cell-specific signature (CSS) comprising at least one unbalanced process active in each cell of the plurality of cells; and
n. assigning cells with the same CSS to a cellular population;
   thereby identifying at least one subpopulation within a population of cells.

By another aspect, there is provided a computer program product, comprising a non-transitory computer-readable storage medium having program code embodied thereon, the program code executable by at least one hardware processor to perform a method of the invention.

By another aspect, there is provided a computer program product, comprising a non-transitory computer-readable storage medium having program code embodied thereon, the program code executable by at least one hardware processor to
o. receive a single-cell expression analysis of the population of cells;
p. calculate for the population of cells at least one unbalanced process active in at least one cell of the population of cells;
q. generate for a plurality of cells of the population of cells a cell-specific signature (CSS) comprising at least one unbalanced process active in each cell of the plurality of cells; and
r. assign cells with the same CSS to a cellular population.

In some embodiments, the method is an in vitro method. In some embodiments, the method is an ex vivo method. In some embodiments, the method is performed at a given timepoint. In some embodiments, the method is identifying a subpopulation present in the population at a given timepoint. In some embodiments, the given timepoint is a single timepoint. In some embodiments, the method is for identifying at least one subpopulation within a cancer. In some embodiments, the cancer is a solid cancer. In some embodiments, the solid cancer is a tumor. In some embodiments, the methods of the invention are computerized methods. In some embodiments, the methods of the invention are performed on a computer. In some embodiments, the data provided, and the output of the method are embodied in electronic files.

As used herein, the term “subpopulation” refers to at least one cell within a larger population of cells that has a unique molecular signature that is different from other cells of the population. In some embodiments, a subpopulation is a plurality of cells. In some embodiments, a subpopulation is at least 1, 2, 3, 5, 10, 20, 25, 30, 40, 50, 100, 200, 300, 400, 500 or 1000 cells. Each possibility represents a separate embodiment of the invention. In some embodiments, a subpopulation is at least 2 cells. In some embodiments, a subpopulation shares a unique set of active unbalanced processes. In some embodiments, the molecular signature is a cell-specific signature (CSS) as defined herein.

In some embodiments, the population of cells comprises at least 10,000. In some embodiments, the population of cells comprises at least 20,000. In some embodiments, the population of cells comprises at least 30,000. In some embodiments, the population of cells comprises at least 40,000. In some embodiments, the population of cells comprises at least 50,000 cells. In some embodiments, the population of cells comprises at least 10000, 20000, 30000, 40000, or 50000 cells. Each possibility represents a separate embodiment of the invention. In some embodiments, the population of cells is the cells of a biopsy. In some embodiments, the biopsy is a fine needle biopsy. In some embodiments, the subpopulation is at least 0.001, 0.005, 0.01, 0.05, 0.1, 0.5, or 1% of the population. Each possibility represents a separate embodiment of the invention.

In some embodiments, the population of cells is from a sample. In some embodiments, the sample is a biological sample. In some embodiments, the sample is a sample comprising cells. In some embodiments, the sample is from a subject. In some embodiments, the sample is a sample from a cancer. In some embodiments, the sample is from a cancer. In some embodiments, the sample is a biopsy. In some embodiments, the biopsy is a tumor biopsy. In some embodiments, the population of cells is cells from a cancer. In some embodiments, the cancer is a solid cancer. In some embodiments, the population of cells is derived from a cancer. In some embodiments, the cancer is a caner in a subject. In some embodiments, the cancer is a cancer of a model organism. In some embodiments, the model organism bares human cancer cells. In some embodiments, human cancer cells are a human tumor. In some embodiments, the model organism bares a model for a human cancer or tumor. In some embodiments, the cancer is a cell line. In some embodiments, the cancer is a tumor cell line. In some embodiments, the cell line is a human cell line.

In some embodiments, the cancer is a human cancer. In some embodiments, the cancer is a solid cancer. In some embodiments, the cancer is a metastatic cancer. In some embodiments, the cancer is a heterogeneous cancer. In some embodiments, the cancer comprises intercellular heterogeneity. In some embodiments, the cancer is breast cancer. In some embodiments, the breast cancer is triple negative breast cancer. Examples of solid cancers include, but are not limited to, lung cancer, head and neck cancer, skin cancer, bladder cancer, gastric cancer, colorectal cancer, ovarian cancer, brain cancer, testicular cancer, and breast cancer. In some embodiments, the cancer is lung cancer. In some embodiments, the cancer is head and neck cancer. In some embodiments, the cancer is brain cancer. In some embodiments, the brain cancer is Glioblastoma. In some embodiments, the cancer is a cancer treatable by radiotherapy. In some embodiments, the cancer is a cancer treatable by chemotherapy. In some embodiments, the cancer is a cancer treatable by a non-targeted cancer therapy.

In some embodiments, the expression data is embodied in an electronic file. In some embodiments, the expression analysis is embodied in an electronic file. In some embodiments, the expression analysis comprises expression data. In some embodiments, the expression is RNA expression. In some embodiments, the expression is transcriptional expression. In some embodiments, the RNA is mRNA. In some embodiments, the expression is protein expression. In some embodiments, the expression is protein expression, mRNA expression or both. In some embodiments, the protein is surface protein. In some embodiments, the protein is secreted protein. In some embodiments, the protein is total protein. In some embodiments, protein expression comprises post-translational protein modification levels. In some embodiments, the protein is a post-translationally modified protein. In some embodiments, the analysis is a single-cell analysis. It will be understood by a skilled artisan that a single-cell analysis provides expression levels for individual cells and not cumulative expression for multiple cells or for the entire population. Single cell proteomic or transcriptional analysis is well known in the art. Any type of single cell expression analysis may be used. In some embodiments, a proteomic analysis comprises FACS analysis. In some embodiments, the FACS analysis is single-cell FACS analysis. In some embodiments, the FACS analysis is surface protein analysis. In some embodiments, the FACS analysis is intracellular FACS analysis. In some embodiments, the receiving is at a given timepoint. In some embodiments, the single-cell expression analysis is analysis of expression at a given timepoint. In some embodiments, the single-cell analysis is provided as a 2D matrix. In some embodiments, the expression analysis is provided as a 2D matrix. In some embodiments, the input for the analysis is a 2D matrix. In some embodiments, the input for the thermodynamic-based analysis is a 2D matrix.

In some embodiments, the expression analysis is analysis of a plurality of proteins characteristic of the population of cells. In some embodiments, the expression analysis is analysis of a plurality of oncogenic or tumor suppressor proteins and/or genes. In some embodiments, the expression analysis is analysis of a plurality of oncogenic proteins and/or genes. In some embodiments, the oncogenic protein/genes, tumor suppressor proteins/genes or both are characteristic of a cancer. In some embodiments, the oncogenic protein/genes are characteristic of a cancer. In some embodiments, the cancer is the cancer from which the population of cells is derived. In some embodiments, they are characteristic of tumors of the solid cancer.

In some embodiments, the expression analysis analyzes a plurality of proteins/genes. In some embodiments, the expression analysis analyzes at least 2, 3, 4, 5, 6, 7, 8, 9, 10, or 11 proteins/genes. Each possibility represents a separate embodiment of the invention. In some embodiments, the expression analysis analyzes at least 3 proteins/genes. In some embodiments, the expression analysis analyzes at least 5 proteins/genes. In some embodiments, the expression analysis analyzes at least 10 proteins/genes. In some embodiments, the expression analysis analyzes at least 11 proteins/genes.

In some embodiments, the method further comprises obtaining the population of cells. In some embodiments, the method further comprises subjecting the population of cells to a single-cell expression analysis. In some embodiments, the receiving comprises obtaining the population of cells and subjecting it to a single-cell expression analysis. In some embodiments, obtaining the population comprises obtaining a sample comprising cells. In some embodiments, the receiving is receiving a single-cell expression analysis of a population of cells derived from the cancer. In some embodiments, the population is converted into a single cell suspension before the expression analysis. In some embodiments, the sample is converted into a single cell suspension before the expression analysis. In some embodiments, the converting is digesting.

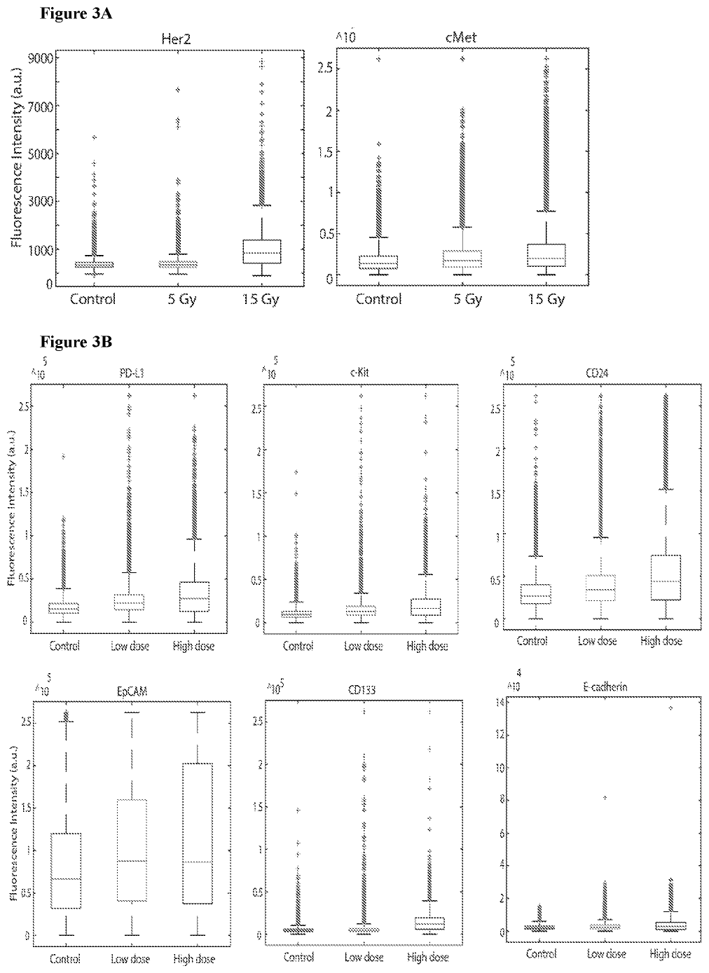

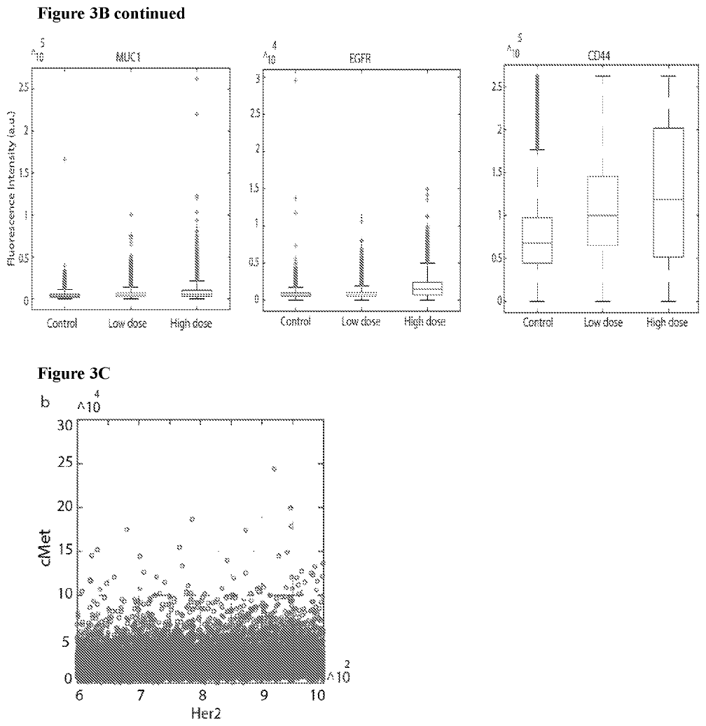

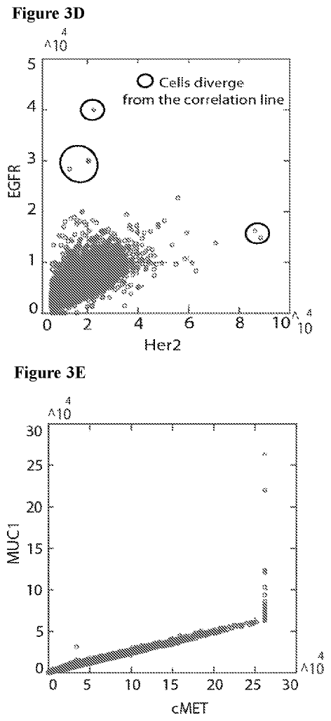

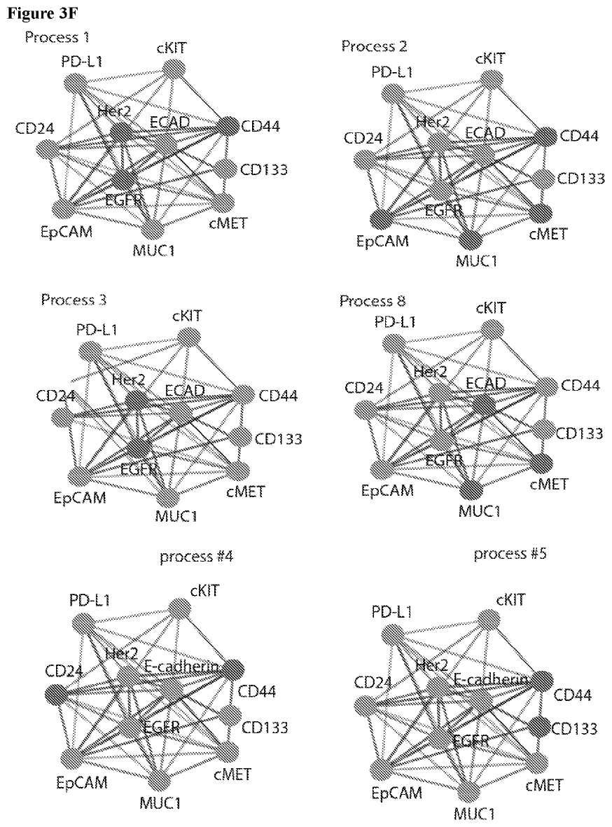

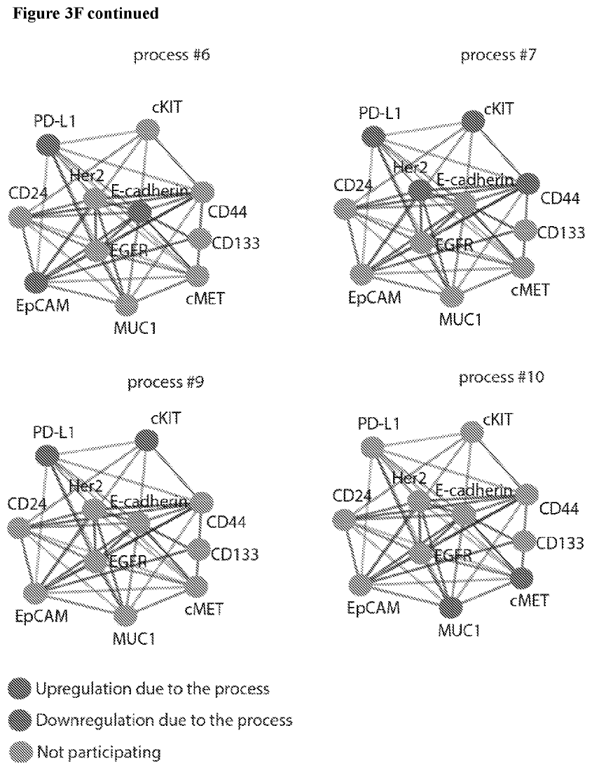

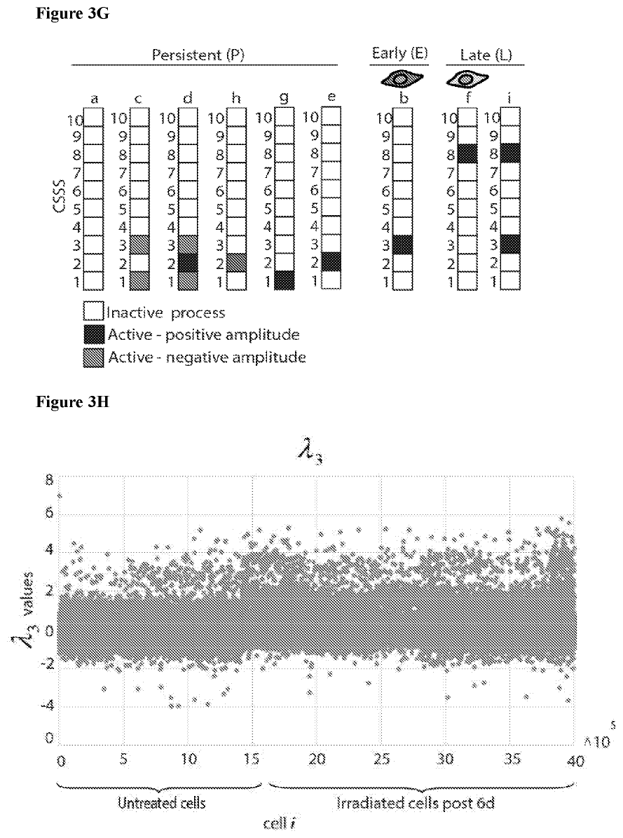

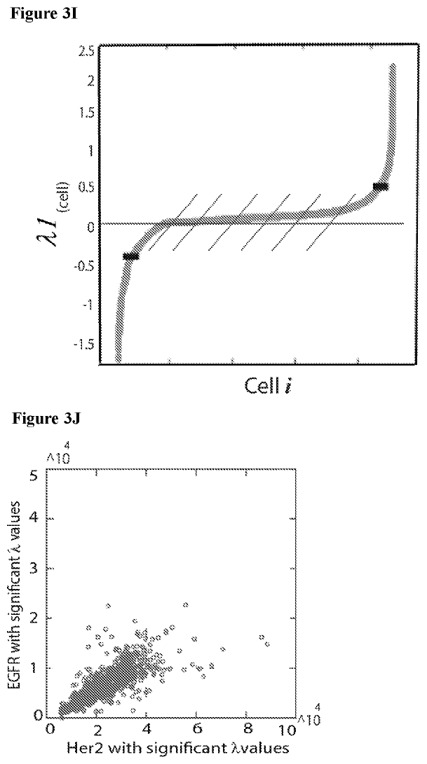

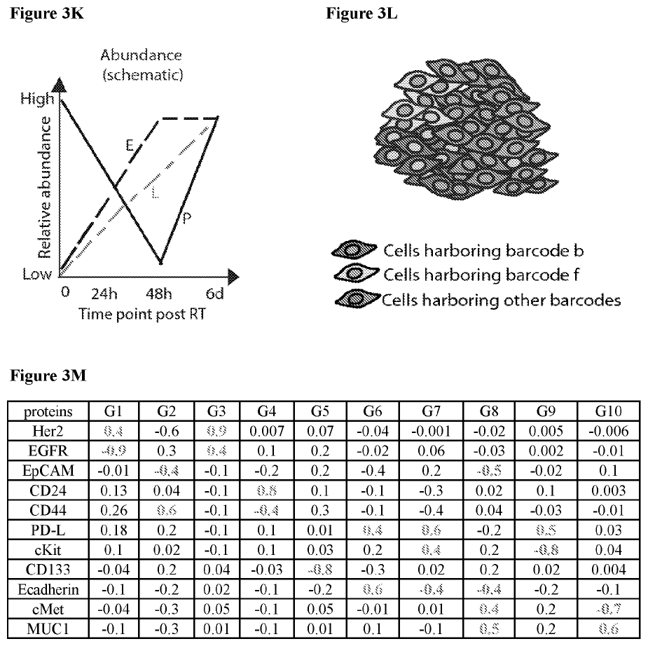

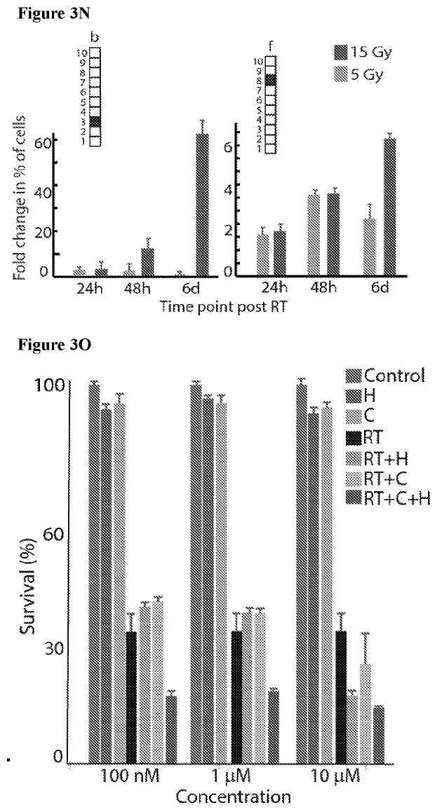

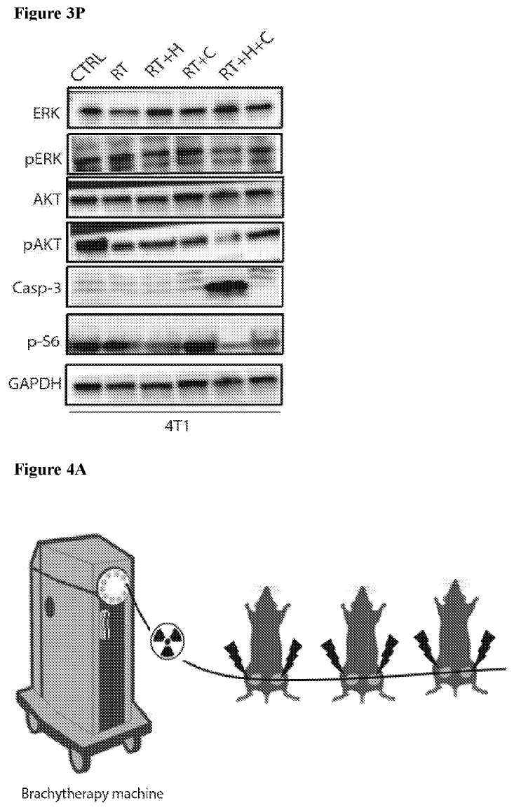

In some embodiments, calculating is determining. The terms “calculating” and “determining” are used herein are used interchangeably. In some embodiments, the calculating is by a thermodynamic-based analysis of the expression analysis. In some embodiments, the calculating comprises performing a thermodynamic-based analysis of the expression analysis. In some embodiments, the thermodynamic-based analysis is a deterministic analysis. In some embodiments, the thermodynamic-based analysis is an information-theoretical analysis. In some embodiments, a thermodynamic-based analysis is an analysis of free energy. In some embodiments, the thermodynamic-based analysis comprises determining processes comprising the proteins/genes analyzed in the single-cell expression analysis. In some embodiments, the thermodynamic-based analysis comprises determining the free energy state of a process. In some embodiments, a process with a minimal free energy is a balanced process. In some embodiments, minimal free energy is maximal entropy. In some embodiments, a process with a free energy above the minimum is an unbalanced process. In some embodiments, the free energy of the process is the amplitude of the process. In some embodiments, the free energy above the minimum is calculated using an amplitude of the process. In some embodiments, a process with statistically significant increase in free energy above the minimum is an unbalanced process. In some embodiments, a process with a statistically significant amplitude is an unbalanced process. In some embodiments, a significant amplitude is an amplitude above a predetermined threshold. In some embodiments, a significant increase is an increase above a predetermined threshold. In some embodiments, the predetermined threshold is as calculated in FIG. 3I. Determination of processes that are active in the cells or proteins significantly upregulated or downregulated can be done based on sigmoid plots: values located on the tails of the plot are considered as significant. Based on these values the cell specific signaling barcodes are calculated.

In some embodiments, the method further comprises an error calculation step. In some embodiments, significant proteins/genes are confirmed with an error calculation step. In some embodiments, determining proteins/genes that participate in unbalanced processes comprises an error calculation step. Methods of error calculation are well known in the art and include for example the method recited in Vasudevan et al., 2018, “Personalized disease signatures through information-theoretic compaction of big cancer data”, herein incorporated by reference in its entirety. Calculation of error limits can be based on the fluctuations in the expression levels of the most stable proteins to determine which of the processes possess an amplitude that exceeds the noise threshold. Further, to validate that the number of significant unbalanced processes (only those having amplitude values exceeding error limits) is sufficient, these processes are verified to adequately reproduce the experimental data. This error calculation allows for even more accurate identification of the processes/active process in each cell and thus the identification of the CSS and subpopulations.

In some embodiments, the thermodynamic-based analysis comprises surprisal analysis. In some embodiments, the thermodynamic-based analysis is surprisal analysis. As used herein, “surprisal analysis” refers to an analysis technique that determines thermodynamic and entropic balanced and unbalanced states in a system. In some embodiments, the surprisal analysis comprises the analysis described herein. In some embodiments, the surprisal analysis comprises using equation [1].

As used herein, a “balanced process” refers to a network of genes/proteins that exists in the sample at maximal entropy or thermodynamic equilibrium. Thus, a balanced process is a network in a balanced state. As used herein, an “unbalanced process” refers to a network of genes/proteins that deviates from the balanced state. This is a network that deviates from thermodynamic steady state. In some embodiments, a process is a signaling network. In some embodiments, a process is a signaling pathway. In some embodiments, a process is a functional pathway. In some embodiments, a process is a functional network. In some embodiments, a process comprises genes/proteins measured in the single-cell expression analysis. In some embodiments, a process consists of genes/proteins measured in the single-cell expression analysis.

In some embodiments, determining at least one unbalanced process comprises determining over and under expressed genes/proteins in each cell's expression data. In some embodiments, the over and under expression is as compared to a control data set or control cell. In some embodiments, the over and under expression is as compared to the average expression in the population. In some embodiments, the over and under expression is as compared to the median expression in the population. In some embodiments, the over and under expression is as compared to other genes/proteins within an unbalanced process. In some embodiments, the over and under expression is as compared to other genes/proteins within the process being examined. A skilled artisan will appreciate that when a process is examined for being balanced or unbalanced a single gene/protein can be determined to be over or under expressed relative to the expression of the other genes/proteins of the process.

In some embodiments, determining at least one unbalanced process comprises assembling expressed genes and/or proteins into networks. In some embodiments, the networks are assembled from genes/proteins from the single-cell expression analysis. In some embodiments, the networks are functional networks. In some embodiments, the assembling is performed using functional interactions. In some embodiments, the function interactions are according to the STRING database.

In some embodiments, at least one unbalanced process is identified in a cell of the population's expression data. In some embodiments, at least one unbalanced process is identified in a plurality of cells of the population's expression data. In some embodiments, at least one unbalanced process is identified in all cells of the population's expression data. In some embodiments, all unbalanced processes are identified in the cells of the population's expression data. In some embodiments, all unbalanced processes that exist in a cell of the population's expression data are identified. In some embodiments, the at least one unbalanced process is selected from the processes provided in FIG. 3F. In some embodiments, the at least one unbalanced process is selected from Table 3.

In some embodiments, determining at least one unbalanced process is determining at least two unbalanced processes. In some embodiments, determining at least one unbalanced process is determining a plurality of unbalanced processes. In some embodiments, determining at least one unbalanced process is determining at least three unbalanced processes. In some embodiments, determining at least one unbalanced process is determining at least four unbalanced processes. In some embodiments, determining at least one unbalanced process is determining at least five unbalanced processes.

In some embodiments, the unbalanced process is active in at least one cell of the population. In some embodiments, the unbalanced process is active in at least a plurality of cells of the population. In some embodiments, the unbalanced process is active in at least 0.001, 0.005, 0.01, 0.05, 0.1, 0.5, or 1% of cells of the population. Each possibility represents a separate embodiment of the invention. As used herein, the term “active” refers to a process that is unbalanced in the cell. A process can be unbalanced or balanced. If the process is unbalanced in a cell, then the unbalanced process is active in the cell. It will be understood by a skilled artisan that there may be processes that comprise proteins/genes measured in the analysis that are not unbalanced in any of the cells of the population. Thus, these processes are not active in any of the cells. Conversely, there may be processes active in a cell of the population, but that comprises proteins/genes not measured in the analysis. Thus, those these processes are indeed active they cannot be determined. The method described herein calls for identifying at least one process that is based on the expression analysis and is unbalanced/active in at least one cell of the population. In some embodiments, calculating at least one unbalanced process active in at least one cell of the population is calculating all unbalanced processes active in at least one cell of the population. In some embodiments, calculating at least one unbalanced process active in at least one cell of the population is calculating all unbalanced processes active in a plurality of cells of the population. In some embodiments, calculating at least one unbalanced process active in at least one cell of the population is calculating all unbalanced processes active in all cells of the population. In some embodiments, all active unbalanced processes are all unbalanced process as based on the thermodynamic-based analysis. In some embodiments, all active unbalanced process is all unbalanced process as based on the expression analysis. It will be understood that “all unbalanced processes” does not refer to unbalanced processes that are based on proteins/genes that were not a part of the analysis. In some embodiments, all unbalanced processes are all unbalanced processes present in a list of calculated unbalanced processes.

In some embodiments, the calculating produces a list of calculated unbalanced process. In some embodiments, the list comprises at least 1, 2, 3, 4, 5, 6, 7, 8, 9 or 10 unbalanced processes. In some embodiments, the list comprises at least a plurality of unbalanced processes. In some embodiments, the list comprises the calculated unbalanced processes present in the population or plurality of cells at a given time. In some embodiments, the list comprises the calculated unbalanced processes present in a given population or plurality of cells.

In some embodiments, the CSS comprises at least one unbalanced process active in a cell of the plurality of cells. In some embodiments, the CSS comprises at least one unbalanced process active in a cell of the population of cells. In some embodiments, the CSS comprises at least one unbalanced process from the list of calculated unbalanced processes. In some embodiments, the CSS comprises unbalanced process from the list of calculated unbalanced processes. In some embodiments, the CSS comprises all unbalanced process active in a cell of the plurality of cells. In some embodiments, the CSS comprises all unbalanced process active in a cell of the population of cells. In some embodiments, the CSS comprises at least one unbalanced process active in each cell of the plurality of cells. In some embodiments, the CSS comprises at least one unbalanced process active in each cell of the population of cells. In some embodiments, the CSS comprises all unbalanced process active in each cell of the plurality of cells. In some embodiments, the CSS comprises all unbalanced process active in each cell of the population of cells. In some embodiments, the CSS is generated for at least one cell of the plurality of cells. In some embodiments, the CSS is generated for each cell of the plurality of cells. In some embodiments, the CSS is generated for at least one cell of the population of cells. In some embodiment, the CSS is generated for each cell of the population of cells. In some embodiments, the CSS is generated from the list of calculated unbalanced processes. In some embodiments, a given cell's CSS comprises all unbalanced processes from the list of calculated unbalanced processes active in the given cell.

In some embodiments, the CSS is a processes barcode. In some embodiments, the CSS is an unbalanced processes barcode. In some embodiments, the methods of the invention comprise assigning to a sample a barcode. In some embodiments, the barcode indicates the unbalanced processes active in the cell. In some embodiments, the barcode indicates the status of all processes in the plurality of cells or population of cells within a given cell.

In some embodiments, the CSS comprises significantly unbalanced processes. In some embodiments, significant is statistically significant. In some embodiments, significant is at least one standard deviate above the balanced state. In some embodiments, a significantly unbalanced process is a process with a significant amplitude. In some embodiments, the CSS comprises all significantly unbalanced processes. In some embodiments, the CSS comprises all significantly unbalanced processes active within a given cell. In some embodiments, an active process is a process that is significantly unbalanced.

In some embodiments, cells with the same CSS are assigned to a cellular population. In some embodiments, a plurality of cells with the same CSS are assigned to a cellular population. In some embodiments, all cells with the same CSS are assigned to a cellular population. In some embodiments, cells with the same CSS are assigned to the same cellular population. It will be understood that two CSSes may have a single process in common but will differ with regards to another process. Thus, each CSS is unique and so each cellular population is unique.

In some embodiments, the method is a method of selecting a therapy. In some embodiments, the method is a method of selecting a therapy for a cancer. In some embodiments, the method is a method of selecting a therapy for treating a cancer. In embodiments in which the method is for selecting a therapy for treating a cancer, the population of cells is derived from the cancer. In some embodiments, the method is a method for selecting a subject-specific therapy and the cancer is a tumor of the subject. In some embodiments, the method is a method for selecting a general anti-cancer therapy and the cancer is a tumor of a model organism. In some embodiments, the model organism bares a human cancer. In some embodiments, the method is a method for selecting a general anti-cancer therapy and the cancer is a model for a human cancer. In some embodiments, the method is a method for selecting a general anti-cancer therapy and the cancer is a cancer cell line. In some embodiments, the cancer is a tumor. In some embodiments, the cancer is a human cancer.

In some embodiments, a method for selecting a therapy comprises performing a method of identifying at least one subpopulation and selecting a therapy that targets the at least one cellular population. In some embodiments, the method comprises selecting a therapy that targets a CSS of the at least one cellular population. In some embodiments, targeting a CSS is targeting a protein/gene of the CSS. In some embodiments, selecting a therapy that targets the at least one cellular population is selecting a therapy that targets a CSS of the at least one cellular population. In some embodiments, the therapy targets at least one protein/gene of the CSS. In some embodiments, the therapy targets at least one protein/gene expressed in the cellular population. In some embodiments, the at least one gene/protein is a druggable target. In some embodiments, the therapy is a drug for the druggable target. In some embodiments, the therapy or drug is a known therapy or drug. In some embodiments, the therapy or drug is an anticancer therapy or drug.

As used herein, a “druggable target” refers to any gene or protein whose expression or function can be modified by administration of a drug. Potential drugs can be selected from any known drug list, or database, including but not limited to the FDA approved drug list, the National Cancer Institute drug list (cancer.gov/about-cancer/treatment/drugs), and drugs.com. In some embodiments, the drug effects only the druggable target. In some embodiments, the drug effects more than one target including the druggable target. Examples of druggable targets and their drug include, but are not limited to, Her2 and Herceptin and cMet and Crizotinib.

In some embodiments, the method further comprises administering the selected therapy. In some embodiments, the administering is to a subject. In some embodiments, the subject is a human. In some embodiments, the subject is a subject suffering from the cancer. In some embodiments, the subject is the subject that provided the population of cells. In some embodiments, the subject is the subject that provided the sample comprises the population of cells. In some embodiments, the subject is a subject that comprises the cancer from which the population of cells was derived. In some embodiments, the subject is the subject that provided the cells derived from a cancer.

As used herein, the terms “administering,” “administration,” and like terms refer to any method which, in sound medical practice, delivers a composition containing an active agent to a subject in such a manner as to provide a therapeutic effect. Suitable routes of administration can include, but are not limited to, oral, parenteral, subcutaneous, intravenous, intramuscular, or intraperitoneal.

The dosage administered will be dependent upon the age, health, and weight of the recipient, kind of concurrent treatment, if any, frequency of treatment, and the nature of the effect desired.

In some embodiments, the population of cells has been contacted by a first therapy prior to the proteomic analysis. In some embodiments, the cancer from which the population of cells is derived has been contacted by a first therapy. In some embodiments, the cancer is from a subject and the subject has been administered the first therapy prior to derivation of the population of cells. In some embodiments, the method is a method of selecting a second therapy. In some embodiments, the method is a method of selecting a combination therapy. In some embodiments, a combination therapy is a combination of the first therapy and the second therapy. In some embodiments, the combination is a combination of the first therapy and at least the second therapy. In some embodiments, the method is a method of selecting at least a second therapy. In some embodiments, the second therapy is a combination of therapies wherein each therapy targets a different cellular population. In some embodiments, a combination therapy is coadministration of the first and the second therapy. In some embodiments, a combination therapy is pre-administration of the second therapy before administering the first therapy. In some embodiments, a combination therapy is pre-administration of the first therapy before administering the second therapy.

In some embodiments, prior comprises a period of time sufficient for regrowth of the cancer. In some embodiments, prior comprises a period of time sufficient for regrowth of the tumor. It will be understood by a skilled artisan that after administration of a first therapy to a subject afflicted with cancer, a model animal comprising a human cancer, a human cancer model or a cancer cell line will have an anticancer effect that will kill many of the cancer cells. However, when a cancer returns/a subject relapses the cells that were not killed by the first therapy may have returned. These cells may be resistant to the first therapy and thus the cancer will be refractory to that first therapy. The instant application shows that the populations of cells that are resistant to the first therapy may be very small, making up less than 1% of the cells of the cancer before the first therapy. These populations however, become evident after regrowth following the first treatment, thus the analysis to find the second therapy should be performed after a time sufficient for these populations to grow up and for at least partial regrowth of the cancer. In some embodiments, prior comprises a period of time sufficient for growth of the cancer to allow obtaining a number of cells needed to perform the method of the invention. In some embodiments, the time is a time sufficient for acquiring at least 50,000 cells for performance of a method of the invention. In some embodiments, the time is a time sufficient for regrowth of at least 20%, 30%, 40%, 50%, 60%, 70%, 80%, 90%, 95% or 100% of the cancer or tumor. Each possibility represents a separate embodiment of the invention. In some embodiments, the time is a time sufficient for regrowth of at least 100% of the tumor. In some embodiments, the time is a time sufficient for regrowth of at least 90% of the tumor. In some embodiments, the time is a time sufficient for regrowth of at least 80% of the tumor. In some embodiments, a percentage of the cancer of tumor is as compared to the tumor before the first treatment. In some embodiments, the percent of the tumor is percent of the volume of the tumor. In some embodiments, the period of time is at least 1, 2, 3, 4, 5, 6, 7, 10, 12, 14, 16, 18, 20, 21, 22, 24, 26, 28, 30, 60, 90, 120, 150, 180, 210, 240, 270, 300, 320, 340, or 365 days. Each possibility represents a separate embodiment of the invention. In some embodiments, the period of time is at least 6 days.

In some embodiments, the method further comprises receiving a single-cell expression analysis of a population of cells derived from a cancer before treatment with the first therapy. In some embodiments, the expression analysis of the population before treatment and the population after is the same analysis. In some embodiments, the analysis before and the analysis after comprises analysis of the same proteins/genes. In some embodiments, the analysis before and the analysis after treatment are both protein analysis. In some embodiments, the method further comprises assigning cells of a plurality of cells of the population of cells derived from the cancer before treatment to a cellular population. In some embodiments, the assigning is assigning all cells of the plurality of cells to a cellular population. In some embodiments, the assigning is assigning all cells of the population of cells to a cellular population. In some embodiments, the assigning comprises calculating for the population of cells derived before treatment at least one unbalanced process active in at least one cell of the population of cells derived before treatment. In some embodiments, the calculating comprises calculating at least two active unbalanced processes. In some embodiments, the calculating comprises calculating all unbalanced process active in the cells of the population of cells derived before treatment. In some embodiments, all unbalanced process is based on the expression analysis of the population of cells derived from the cancer before treatment with the first therapy. In some embodiments, the calculating before the therapy and after the therapy are the same calculating. In some embodiments, the thermodynamic-based analysis on cells before the first therapy is the same thermodynamic-based analysis of cells after the first therapy. In some embodiments, the calculating comprises producing a list of calculated unbalanced process active in the population of cells before the first therapy. In some embodiments, the calculating comprises generating for the plurality of cells a CSS. In some embodiments, the calculating comprises generating for each cell of the plurality of cells a CSS. In some embodiments, the CSS comprises at least one unbalanced process from the list of calculated unbalanced processes that is active in a given cell of the population of cells derived from the cancer before treatment. In some embodiments, the CSS comprises all unbalanced process from the list of calculated unbalanced processes that are active in a given cell of the population of cells derived from the cancer before treatment. In some embodiments, the method further comprises assigning at least two cells with the same CSS to a cellular population. In some embodiments, the method further comprises assigning all cells with the same CSS to a cellular population.

In some embodiments, an increase in abundance is at least a 1, 2, 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 600, 700, 800, 900, or 1000% increase in abundance. Each possibility represents a separate embodiment of the invention. In some embodiments, an increase is a statistically significant increase. In some embodiments, an increase is an increase above a predetermined threshold. In some embodiments, the threshold is determined as shown in FIG. 3I. In some embodiments, the method further comprises calculating the percent of all cells of the population before treatment that are in each cellular population. In some embodiments, the method further comprises calculating the percent of all cells in the population after treatment that are in each cellular population. In some embodiments, the method further comprises comparing for at least one population abundance before treatment with the abundance after treatment. In some embodiments, an increase is an increase is absolute abundance. In some embodiments, an increase is an increase in relative abundance as compared to the total population. In some embodiments, an increase in abundance of a cellular population is an increase in the percent the population is of the total

In some embodiments, the selecting a second therapy is selecting a second therapy that targets at least one cellular population that increased in abundance following the first therapy. In some embodiments, the selecting a second therapy is selecting a second therapy that targets a CSS of at least one cellular population that increased in abundance following the first therapy. In some embodiments, the selecting a second therapy is selecting at least a second therapy that targets at least two cellular populations that increased in abundance following the first therapy. In some embodiments, the selecting a second therapy is selecting at least a second therapy that targets a plurality of cellular populations that increased in abundance following the first therapy. In some embodiments, the at least a second therapy is a plurality of therapies. In some embodiments, the second therapy targets all populations that increased in abundance. In some embodiments, the second therapy targets the population that increases the most in abundance. In some embodiments, the second therapy targets the most abundant population following the first therapy. In some embodiments, the second therapy targets the two most abundant populations following the first therapy. In some embodiments, the second therapy targets the most abundant population that increased after the first therapy. In some embodiments, the second therapy targets all populations that increased in abundance and are at least 1, 2, 4, 5, 10, 20, 25, 30, 35, 40, 45, or 50% of the total population following the first therapy. Each possibility represents a separate embodiment of the invention. In some embodiments, the second therapy targets all populations that increased in abundance and are at least 1% of the total population following the first therapy. In some embodiments, the second therapy targets all populations that increased in abundance and are at least 2% of the total population following the first therapy.

In some embodiments, the first therapy is an untargeted therapy. In some embodiments, the first therapy is an untargeted cancer therapy. In some embodiments, the untargeted therapy is radiotherapy. In some embodiments, radiotherapy is irradiation. In some embodiments, the untargeted therapy is chemotherapy. A skilled artisan will understand that the dose and intensity of the irradiation and/or the dose of chemotherapy will be according to the accepted dosing regime and best practice for treatment of a given cancer. In some embodiments, the untargeted therapy is an immune cell transfer. In some embodiments, the untargeted therapy is an adoptive immune cell transfer. In some embodiment, the immune cell is a T cell. In some embodiments, the first therapy is selected from radiotherapy and chemotherapy. In some embodiments, the first therapy is selected from radiotherapy, immune cell transfer and chemotherapy.

In some embodiments, the second therapy is a targeted therapy. In some embodiments, the second therapy targets a CSS. In some embodiments, the second therapy targets a protein of a CSS. In some embodiments, the second therapy targets a protein of an unbalanced process of a CSS. In some embodiments, the second therapy targets a druggable target protein of a CSS or an unbalanced process of a CSS. In some embodiments, the second therapy targets a protein of an unbalanced process active in the cellular population being targeted. Example of targeted therapies are well known in the art and can be selected based on the desired protein/process to be targeted. For a non-limiting example, targeting of Her2 can be carried out with Herceptin or trastuzumab, targeting of cMet can be carried out with Crizotinib, targeting of estrogen receptor may be carried out with Tamoxifen, targeting of EGFR may be carried out with Erlotinib or lapatinib, targeting of VEGFR2 may be carried out with ramucirumab, targeting of Src may be carried out with dasatinib, and targeting of Braf may be carried out with vemurafenib, to name but a few. It will be understood by a skilled artisan that if a protein/gene is overexpressed or upregulated in an unbalanced process then targeting the protein/gene would comprises inhibiting its expression or function; whereas, if a protein/gene is under-expressed or downregulated in an unbalanced process then targeting the protein/gene would comprise activating or enhancing its expression or function.

In some embodiments, the method further comprises administering the second therapy. In some embodiments, the method further comprises administering the combination therapy. In some embodiments, the method further comprises administering the first and second therapy. In some embodiments, the administering is to a subject. In some embodiments, the subject is a subject suffering from the cancer. In some embodiments, the subject provided the population of cells derived from the cancer. In some embodiments, the subject has the cancer from with the population of cells is derived. In some embodiments, the subject comprises the physical cancer from with the population of cells is derived. In such embodiments, the first therapy, second therapy, combination therapy or a combination thereof are patient-specific therapies.

According to another aspect, there is provided a method of treating a subject suffering from breast cancer, the method comprising administering to the subject a first therapy selected from radiotherapy or chemotherapy and at least one second therapy selected from anti-Her2 therapy and anti-cMet therapy, thereby treating triple-negative breast cancer.

In some embodiments, the breast cancer is triple-negative breast cancer (TNBC). In some embodiments, the breast cancer is Her2 negative breast cancer. In some embodiments, the breast cancer does not strongly express Her2. In some embodiments, the breast cancer is estrogen receptor negative breast cancer. In some embodiments, the breast cancer is progesterone receptor negative breast cancer.

In some embodiments, the first therapy is radiotherapy. In some embodiments, the method comprises administering both an anti-Her2 therapy and an anti-cMet therapy. In some embodiments, the second therapy is anti-Her2 therapy and anti-cMet therapy. In some embodiments, the anti-Her2 therapy and said anti-cMet therapy are administered concomitantly. In some embodiments, the second therapy is administered concomitantly with the first therapy. In some embodiments, the second therapy is administered before the first therapy. In some embodiments, the anti-Her2 therapy is Herceptin. In some embodiments, the antib-cMet therapy is Crizotinib.

In some embodiments, the method further comprises determining within the breast cancer at least one minor cell populations with a CSS targetable by anti-Her2 therapy, anti-cMet therapy or both. In some embodiments, the determining within the breast cancer is at least two minor cell populations, wherein a first cellular population comprises a CSS targetable with anti-Her2 therapy and a second cell population comprise a CSS targetable with anti-cMet therapy. In some embodiments, a cellular population targetable by anti-Her2 therapy comprises a CSS comprising an unbalanced process with overexpression/upregulation of Her2. In some embodiments, a cellular population targetable by anti-cMet therapy comprises a CSS comprising an unbalanced process with overexpression/upregulation of cMet. In some embodiments, the unbalanced process is an active unbalanced process. In some embodiments, the determining is determining that the breast cancer comprises the at least one minor cellular population. In some embodiments, the determining is determining that the breast cancer comprises the at least two minor cellular populations.

In some embodiments, the computer program product outputs the cellular populations. In some embodiments, the computer program product further selects a therapy that targets at least one cellular population. In some embodiments, the computer program product further outputs the selected therapy.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

By another aspect, there is provided a second therapy comprising anti-Her2 therapy or an anti-cMet therapy for use in combination with a first therapy for treating breast cancer.

In some embodiments, the second therapy is anti-Her2 therapy. In some embodiments, the second therapy is anti-cMet therapy. In some embodiments, the first therapy is anti-Her2 therapy and anti-cMet therapy. In some embodiments, the first therapy is selected from radiotherapy and chemotherapy. In some embodiments, the first therapy is radiotherapy. In some embodiments, the treating breast cancer is treating a subject in need thereof. In some embodiments, the treating breast cancer is treating a subject suffering from breast cancer.

By another aspect, there is provided a kit comprising an anti-Her2 therapy and an anti-cMet therapy.

In some embodiments, the kit further comprises a label stating the anti-Her2 therapy and the anti-cMet therapy are for use in combination with a first therapy for treating breast cancer. In some embodiments, the first therapy is radiotherapy. In some embodiments, the kit is for use in combination therapy with a first therapy for treating breast cancer.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods:

Cell lines and culture. Murine 4T1 cell line is a model mimicking stage IV of TNBC. Human TNBC cells MDA-MB-468 and MDA-MB-231 were acquired from ATCC and authenticated by the Genomic Center of the Technion Institute (Haifa). PDX human derived xenograft BR45 were obtained from the Oncology Department at Hadassah-Jerusalem Medical Center obtained with prior written informed consent.

4T1 cells were routinely maintained in Dulbecco's modified Eagle's medium (DMEM) MDA-MB-231 and MDA-MB-468 were maintained in RPMI-1640 medium; supplemented with 10% FBS, 4 mM L-glutamine, 100 U/mL Penicillin and 100 μg/mL Streptomycin. All media and supplements were from Biological Industries, Israel. All cell lines were maintained at 37° C. in 5% CO2. Cells were checked on a routine basis for the absence of mycoplasma contamination.

Irradiation of parental cells: Cells were treated by single radiation with 5, 10, and 15 Gy doses of γ-rays of ⁶⁰Co by a radiotherapy unit (gamma cell 220) at a dose rate of 1.5 Gy/min.

4T1, MDA-MB-231, MDA-MB-468 and Br45 cells were trypsinized and plated to reach optimal confluences next day by (70-80) % before irradiation treatment. The next day, 4T1 cells were irradiated using (5 and 15) Gy of γ-rays. Radiation doses were selected based on calibration experiments in which the survival rates after irradiation ranged from 40-50% of the cells. MDA-MB-231 and BR45 cells were treated with 10 Gy with the exception of MDA-MB-468 cells which were treated with 5 Gy. Afterwards, cells were grown under normal conditions for 24 h, 48 h and 6 days. At each indicated timepoint, cells were detached from the flask using Acutase and fixed using 2% paraformaldehyde for 30 min in ice. Labelling procedure of each condition performed on the day of the flow cytometry analysis as mentioned below.

Mouse models: Syngeneic mouse model: 2.0×10⁵ 4T1 cells were inoculated subcutaneously on 6-7 weeks old female Balb/c mice. Allogeneic model: BR45 tumors were induced on NSG mice either by injecting 4.0×10⁶ cells orthopedically or subcutaneously transplanting xenografts. After reached the desired tumor volume (80-100 mm3), mice were randomly grouped to around 8-10 per cage and treatment were initialized. Tumor sizes were regularly measured with an electronic calliper every two days and their volumes were obtained using the formula V=(W (2)×L)/2. Mice were kept under conventional pathogen-free conditions. All in-vivo experiments were performed with approval of the Hebrew University of Jerusalem IACUC.

Tumor inoculation: 4T1 mouse breast carcinoma mimics stage IV triple negative breast cancer in human. It is highly metastatic to lungs, lymph nodes, liver and bones after implantation in the mammary fat pad of immune-competent Balb/c mice. Primary tumors were harvested after euthanizing female mice using carbon dioxide (CO2) inhalation. Time elapsed from tumor inoculation varied from 2 weeks to 1 month. Human Br45 was used as PDX model, tumors were harvested 2 months after surgically transplanting xenografts on NSG-NOD mouse model or after 4 months from injecting Br45 cells orthopedically.

High dose rate (HDR) brachytherapy: Tumors were irradiated applying brachytherapy afterloader (GammaMed™ HDR, Iridium 192). 12 Gy was applied on two alternative days. The treatment field was designed with the help of MRI imaging to cover the tumors and protect the rest of the body.

Targeted inhibitors: Herceptin (trastuzumab; Her2 inhibitor) was purchased from Teva Pharmaceutical Industries Ltd. Crizotinib (cMet inhibitor, #12087-50) and Erlotinib (#10483-1) were purchased from Cayman Chemical.

Drugs were given usually by IP or gavage depending on the drug. Herceptin was given IP twice a week with a concentration of 5 mg/kg, the vehicle was 200 ul sterile saline. Crizotinib and erlotinib were given by gavage with a concentration of 25 mg/kg and 12.5 mg/kg respectively, five days a week. The vehicle used was hydroxypropyl methylcellulose with 0.2% tween. Mice were treated for 3 weeks. During this period the tumor volumes were measured regularly to observe the action of the drug. The treatment was based on the prediction by Surprisal analysis and the in vitro validation.

FACS Antibodies: The following fluorescently tagged antibodies, were obtained from BioLegend, Inc.: EpCAM (9C4/G8.8), CD45 (2D1/104), CD31(WM95/390), CD140a (16A1/APAS), CD44 (IM7), E-Cadherin (DECMA-1), EGFR (AY13), CD24 (M1/69), CD24 (ML5), KIT (ACK2/104D2), CD133 (315-2C11/clone7), PD-L1 (10F.9G2/29E.2A3). ERBB2/Her2 (5J297) was obtained from LifeSpan BioScience. Anti-MUC1 Polyclonal Antibody and Anti-Met Polyclonal Antibody were both obtained from Bioss Antibodies Inc. (Table 1).

TABLE 1
Antibodies for flow cytometry analysis.

| | | | | | Concentration |
|---|---|---|---|---|---|
| Protein | Reactivity | Color | Company | Cat. # | (μl/8 × 10⁵ cells) |
| CD45 | Mouse (M) | PE | Biolegend | 103105 | 0.2 |
| CD140 | M | PE | Biolegend | 135905 | 1 |
| CD31 | M | PE | Biolegend | 102407 | 0.3 |
| CD326 - EpCAM | M | APC | Biolegend | 118213 | 1 |
| CD24 | M | BV421 | Biolegend | 101825 | 0.75 |
| CD117 - cKit | M | BV650 | Biolegend | 135125 | 1.5 |
| CD274 - PD-L1 | M | BV605 | Biolegend | 124321 | 1.5 |
| CD133 | M | PE/Dazzel594 | Biolegend | 141211 | 1.5 |
| CD45 | Human (H) | PE | Biolegend | 638512 | 0.2 |
| CD140 | H | PE | Biolegend | 323504 | 1 |
| CD31 | H | PE | Biolegend | 303405 | 0.25 |
| CD326 - EpCAM | H | APC | Biolegend | 324207 | 1 |
| CD24 | H | BV421 | Biolegend | 311121 | 0.75 |
| CD117 - cKit | H | BV650 | Biolegend | 313221 | 1.5 |
| CD274 - PD-L1 | H | BV605 | Biolegend | 329723 | 1.5 |
| CD133 | H | PE/Dazzel594 | Biolegend | 372811 | 1.5 |
| CD44 | H/M | BV510 | Biolegend | 103043 | 1.5 |
| cMet | H/M | Alexa | Bioss | 0668R- | 1.5 |
| | | Fluor 750 | | A750 | |
| MUC1 | H/M | Alexa | Bioss | 1497R- | 1.5 |
| | | Fluor 680 | | A680 | |
| Her2 | H/M | FITC | LifeSpan | C533753 | 1 |
| | | | BioScience | | |
| EGFR | H/M | Per/CP5.5 | Santa Cruz | 20 PCPC5 | 1 |
| CD324 - ECAD | H/M | PE/Cy7 | Biolegend | 147309 | 1.5 |

Preparation of single cell suspensions and flow cytometry analysis: Following mice euthanization, tumors were resected and mechanically disrupted to generate a single-cell suspension. The harvested tumors were washed twice with PBS at RT and minced thoroughly. Then the masses are smashed gently with the back of 10 ml plastic syringe to mechanically digest them. After being placed in the stir apparatus for 15-20 min, the tumor/PBS buffer mixture is to be strained through a 70 μm cell strainer and then centrifuged for 5 min at 3000 rcf. Red blood cells were lysed (15 mM NH4Cl+10 mM KHCO3 for 5 min at R.T.) from the freshly harvested tumors. To stop the reaction of the buffer, 30 ml of DMEM with 10% FBS was added and then the mixture was centrifuged for 5 min at 3000 rcf to get rid of the lysis buffer.

Samples were fixed and permeabilized with 2% PFA (#15710, EMS) for 30 min in ice. 1.5 ml centrifuge tubes, each contains 0.8×10⁶ cells, were incubated for 30 mins in ice with 50 ul of Fc blocker buffer (FACS Buffer+Fc Blocker: anti-mouse CD16/32 Antibody, Biolegend #101301, 1:50).

Each sample was labelled with 11 fluorescently tagged Abs mixture (see antibodies above). A cocktail of 3 additional Abs with the same fluorophore (PE) used as an exclusion criterion for hematopoietic (CD45), fibroblasts (CD140) and endothelial cells (CD31) to ensure that only tumor cells will be analyzed later on. This criterion is not needed in case of staining parental cells which do not have a tumor microenvironment. Unstained control sample for each condition was used along with a single-color control for each Ab using UltraComp Compensation eBeads™ according to the manufacturer's instructions for creating compensation controls. The labelling time extended to 40 min in ice in the dark. Then, samples were washed with an equal amount of flow FACS buffer, centrifuged and resuspended in 700 μl of flow FACS buffer then filtered right before analysis. Cells were analyzed using BD FACS LSR Fortessa. Compensation control was done using UltraComp eBeads (#01-2222-41, ThermoFisher). 50,000 cells were profiled for each sample.

Preliminary data analysis was done using FlowJo VX software. The output data were extracted into an excel file in which each row represented a single cell and each column showed the intensity of each assayed protein (FCS Extract 1.02 software).

Western blot Antibodies: Western blot antibodies were obtained from Cell Signaling Technology, Inc.: anti-phospho-Akt (Thr308, #4056S), anti-phospho-Akt (Ser473, #9271S), anti-total-Akt (#4691S), anti-phospho-ERK1/2 Thr202/Tyr204 (#9101S), anti-total-ERK1/2 (#9102S), anti-cleaved PARP(#5625S), Cleaved Caspase-3 (Asp 175, #9661S), Phospho-S6 Ribosomal Protein (ser235/236, #2211S) (D57.2.2E) XP® Rabbit mAb. GAPDH Antibody (#32233) was obtained from Santa Cruz Biotechnology Inc.

Cell pellets was lysed with a 20% SDS buffer. Protein content of each lysate was determined with a Pierce BCA Protein Assay Kit (#23225, ThermoFisher). Equal protein aliquots were subjected to SDS-PAGE (Criterion Stain Free, 4-15% acrylamide, BIO-RAD) under reducing conditions and proteins were transferred to a nitrocellulose membrane. (Millipore). Membranes were blocked with 5% non-fat milk for 1 hour at R.T. and probed with the appropriate antibody, followed by horseradish peroxidase-conjugated secondary antibody (#123449, Jackson ImmunoResearch) and a chemiluminescent substrate (ECL #170-5061, Bio-Rad).

Survival Analysis: Cells were seeded at 70% confluency and treated as needed for different timepoints. Cells were washed with PBS and fixed with 4% PFA for 10 min. at R.T. The fixed cells were stained with Methylene Blue (MB) for 1 hour at R.T., washed and air dried overnight. The color was extracted with 0.1M HCl for 1 hour at R.T. Absorbance was read at 630 nm.

Data analysis: The analysis composes of two steps. Step 1: single cell surprisal analysis (SA) is utilized to identify unbalanced processes in the cellular population.

Surprisal analysis is a thermodynamic-based information-theoretical approach. The analysis is based on the premise that biological systems reach a balanced state when the system is free of constraints. However, under the influence of environmental and genomic constraints, the system is prevented from reaching the state of minimal free energy, and instead reaches a state which is higher in free energy (in biological systems, which are normally under constant temperature and constant pressure, minimal free energy equals maximal entropy).

For example, if the system under study is a living cell, an environmental constraint can be exposure to a drug, which inflicts a change in protein concentrations and activities in the cell. The system can be influenced by genomic constraints as well, such as genomic mutations that in turn affect protein function, often eliciting alteration of specific signaling pathways to oppose the functions of the damaged protein.

Surprisal analysis can take as input the expression levels of various macromolecules, e.g. genes, transcripts, or proteins. However, be it environmental or genomic alterations, it is the proteins that execute the main functions of a cell, and therefore we base our analysis on proteomic data. The varying forces, or constraints, that act upon living cells ultimately manifest as alterations in the cellular protein network. Each constraint induces a change in a specific part of the protein network in the cells. The subnetwork that is altered due to the specific constraint is termed an unbalanced process. System can be influenced by several constraints thus leading to the emergence of several unbalanced processes. When tumor cells are characterized, the specific set of unbalanced processes can be active in a cell. This is what constitutes the cell-specific signaling signature.

In heterogeneous tissues many processes can occur through the actions of individual cells. Thus, the analysis was implemented independently for each measured cell. The levels of different proteins for each cell at each timepoint t are represented as Equation 1:

$${\underbrace{{X}_{i} ( cell , t )}}_{\begin{matrix}experimental \\ level of protien i\end{matrix}} = {\underbrace{{X}_{i}^{o} ( cell , t )}}_{\begin{matrix}level of protein i \\ in the reference state\end{matrix}} exp {\underbrace{( - {\sum }_{α = 1} {G}_{i α} {λ}_{α} ( cell , t ) )}}_{\begin{matrix}changes in protein levels \\ due to the constraints α = 1 , 2 , …\end{matrix}} \qquad ( 1 )$$

For every protein, i, surprisal analysis calculates the distribution of the expression levels at the reference state: Xᵢ^{O}. This term was shown to be constant, i.e. is independent of time and of the actual state of the system. In terms of information theory, Xᵢ^{O} represents the state of maximal entropy, or minimal information.

Here, Xᵢ⁰ (cell,t) the expected expression level of a protein i at the reference state in a measured cell at the timepoint t. The exponential term in Equation 1 represents the deviation from the reference value due to the constraints, including those imposed by Irradiation. G_{iα} are weights (the degree of participation) of a protein i in the unbalanced processes α=1, 2 . . . .

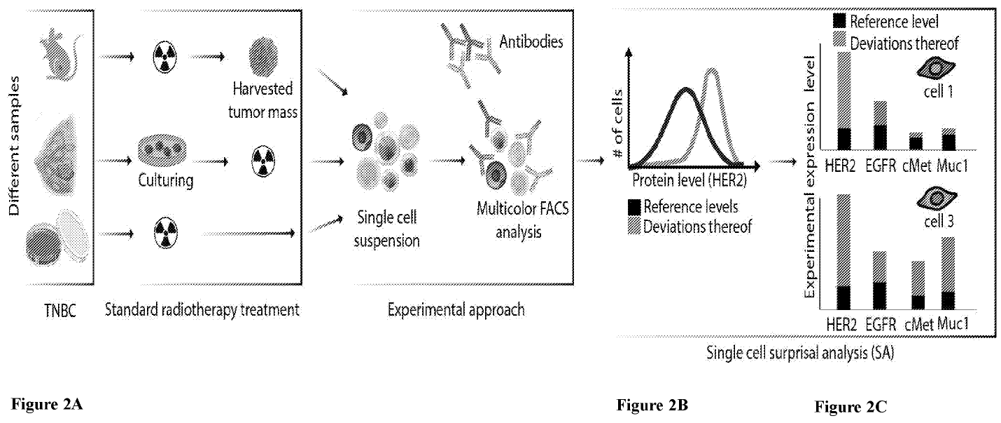

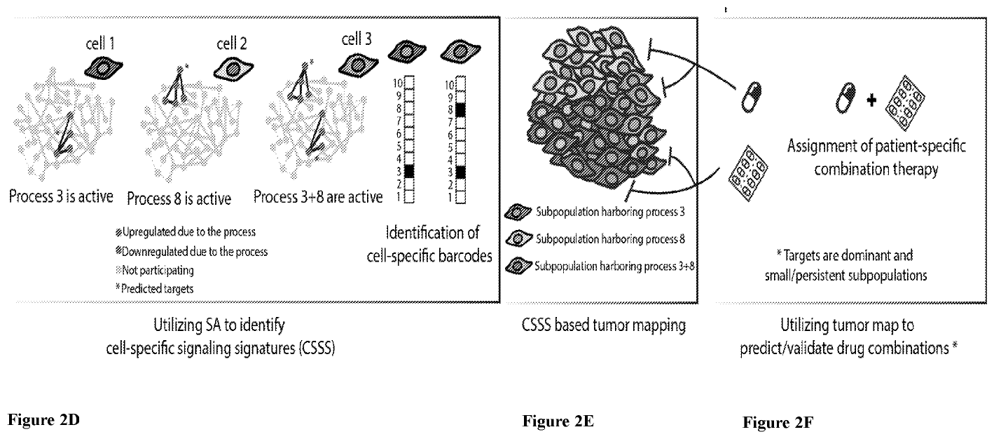

Step 2: To map further distinct subpopulations within the entire cellular population all the cells sharing the same set of unbalanced processes, or CSSS, are grouped into subpopulations (FIG. 2). Each CSSS is transformed into a barcode for the simplicity of calculations and representation.

Proteins deviating in a similar manner from the steady state are grouped into unbalanced processes (FIG. 2). λ_{α}(cell,t) is an amplitude of an unbalanced processes α=1, 2 . . . in a cell i at timepoint t. (Example for G_{iα} values, as calculated for 4T1 models is presented in Table 2. FIG. 3H represents λ₃ (cell,t) values for process 3. Several unbalanced processes can be found in the system, however not all processes are active in all cells.

G_{iα} sign indicates the correlation or anti-correlation between proteins in the same process. For example, in a certain process α, proteins can be assigned the values: G_{protein 1,α}=−0.50, G_{protein 2,α}=0.44, and G_{protein 3,α}=0.00, indicating that this process altered expression levels of the proteins 1 and 2 in opposite directions while not affecting protein 3. Each protein can take part in a number of unbalanced processes at once. Note that in order to define upregulation or downregulation in protein expression levels due to a specific process α the product G_{iα}λ_{α}(cell,t) is calculated.

Importantly, not all processes are active in all cells. The term λ_{α}(cell,t) represents the importance of the unbalanced process α in cell. Its sign indicates the correlation or anti-correlation between the same processes in different cells. For example, if the process α is assigned the values: λ_{α}(1)=3.1, λ_{α}(2)=0.0, and λ_{α}(3)=2.5, it means that this process influences the cells indexed 1 and 3 in the same direction, while it is inactive in cell 2.

Generation of functional networks. FIG. 3F shows the functional networks active in the system. The goal was to generate unbalanced processes composed of proteins with significant G_{iα} values. Functional connections between the proteins in each unbalanced process are based on STRING database.

Barcode calculations: The output lambda file from the surprisal analysis is then used as an input file for the Python script in order to obtain a specific barcode for each single cell in which a certain unbalanced process is active/inactive. The barcodes presented in FIG. 3G were generated using python script. In this script, for each process α, λ_{α}(cell) α=1, 2, 3, . . . , 10 values were normalized as follows: If, e.g.; λ_{α}(cell)>0.5 (and is therefore significant according to calculation of threshold (limit) values) then it was normalized to 1; if λ_{α}(cell)<−0.5 (significant according to threshold values as well) then it was normalized to −1; and if −0.5<λ_{α}(cell)<0.5 then it was normalized to 0. These thresholds (limits) were obtained after λ_{α}(cell) values were sorted according to their values, and only cells with significant λ_{α}(cell) values were considered to possess an unbalanced process α. Only λ_{α}(cell) values located on the tails of the sorted distributions are considered significant. For more details see FIG. 3I.

### Example 1: An Overview of the Integrated Experimental-Computational Approach Used Herein

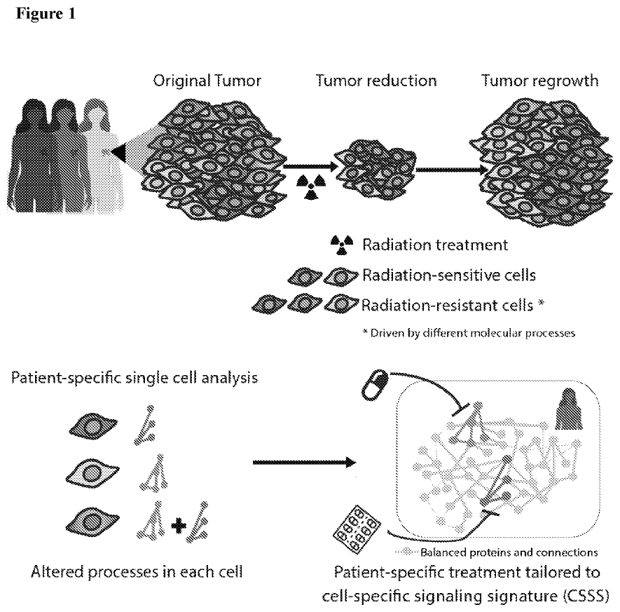

This study was based on the notion that TNBC tumors that undergo irradiation treatment, while initially responding to the treatment, often eventually relapse and regrow (FIG. 1). It was hypothesized that the ability of the tumors to relapse stems from the existence of intra-tumoral subpopulations that do not respond well to the irradiation treatment and drive the regrowth of the tumor post-radiation (FIG. 1, top).

Thus, TNBC tumor composition was studied on the single cell level, with the aim to identify a set of intra-tumoral subpopulations, including very small subpopulations, that demonstrate diminished response to radiation therapy (RT) treatment. By elucidating the altered molecular processes that each subpopulation harbors, a therapeutic strategy is devised that intensifies the response of the tumor to irradiation treatment (FIG. 1, bottom).

Several dimensionality reduction algorithms have been developed to interpret single cell variations (e.g. variations in protein or gene expression levels), such as clustering-based t-SNE analysis and principle component analysis (PCA). These methods are very useful in statistical determination of dominant expression patterns. However, they have limitations when a more deterministic partitioning of the tumor mass into cellular subpopulations, based on cell-specific sets of altered molecular processes, is required. For example, t-SNE is a non-deterministic method (e.g. different runs with same hyper parameters may produce different results) and is unable to assign a certain protein to several processes, or to determine which processes are active in every cell. Therefore, t-SNE will be less efficient when the determination of robust cell-specific signaling signatures is required (e.g. for drug combination design). Similarly, PCA focuses mainly on the most dominant patterns, obtained from proteins with the highest variability in the population, rather than on cell-specific sets of altered processes.

A deterministic approach was sought, in which one can plot every single cell according to its molecular aberrations and network reorganization. To this end, the information theoretic method surprisal analysis (SA) was employed, which was originally applied to characterize the dynamics of non-equilibrium systems in chemistry and physics. This type of analysis has been utilized to quantify bulk proteomic changes in big datasets, including multiple patient tissues or cancer cell lines in order to predict a change in the behavior of the systems or design individualized drug therapies. It has not been used for single cell proteome analysis of various cellular populations.

Herein the approach is extended to quantify the expression changes in single cells, in order to accurately characterize the change in tumor cellular population in response to a first therapy (RT).

This analysis is based on the premise that the application of radiotherapy to TNBC cells induces certain constraints within the tumor mass. These constraints result in altered expression levels of certain proteins, relative to their balanced levels. SA recognizes the constraints operating in the system by identifying groups of proteins that exhibit similar deviations from their balanced state (FIG. 1 and below). A group of proteins demonstrating similar alterations in expression patterns is defined as an unbalanced process. Hence, every constraint that operates on the system gives rise to an unbalanced process.

SA identifies the unbalanced processes that operate in the system under study, including the group of proteins affected by each process. Importantly, each protein is allowed to participate in several processes.

Not all processes are active in all cells, i.e. a certain process can have a negligible amplitude (is balanced) in some cells and a significant amplitude (is unbalanced) in others. A number of different unbalanced processes may operate simultaneously in every cell (FIG. 1). The cell-specific signaling signature (CSSS) is defined for each cell, as the set of active unbalanced processes in the specific cell.

To collect high resolution information regarding the intra-tumoral composition of TNBC tumors, the following experimental technique was employed (FIG. 2): Samples obtained from multiple sources (e.g. cell lines, mouse models and patient-derived tumor cells) were processed to achieve single cell suspensions (FIG. 2A). The cell suspensions were then labeled with fluorescently labeled antibodies targeting selected cell-surface oncoproteins and assayed by multicolor FACS to reveal the accurate expression levels of the labeled proteins in each single cell (FIG. 2A).

In each experimental condition around (30,000-50,000) single cells were profiled. This number of cells allowed for the eventual identification of different subpopulations, including very small subpopulations, comprising less than 1% of the entire population, that are therefore hardly detectable or undetectable in pathological tests.

The selection of the protein panel was based on an extensive literature search to filter oncoproteins that best represent the possible expression patterns in TNBC cells. 11 cell-surface oncoproteins were selected which are involved in breast cancer/cancer stem cell proliferation and represent potential druggable targets for therapy or biomarkers for diagnostics: Her2, EGFR, EpCAM, CD44, CD24, PD-L1, KIT, CD133, E-Cadherin, cMet and MUC1.

Results obtained from FACS measurements were analyzed by SA to reveal proteins which demonstrate deviations in expression levels relative to their balanced state levels (FIG. 2B), and then cell-specific protein-protein correlation expression patterns were examined (FIG. 2C-D), in order to identify the unbalanced processes that have emerged in the cells, as well as the sets of unbalanced processes that operate in specific cells, namely the CSSS (FIG. 2D). Each CSSS is graphically represented by a cell-specific barcode where white squares mean inactive (balanced) processes and black/gray mean active (unbalanced) processes in a cell (FIG. 2D, right panel). Cellular subpopulations are then defined as groups of cells harboring the same CSSS (FIG. 2E)

Note that different subpopulations may share similar processes, e.g. the red and orange subpopulations in FIG. 2 both harbor unbalanced process 3 (FIG. 2D). However, the complete set of unbalanced processes, namely the CSSS, is what defines a subpopulation and governs the therapeutic strategy that should be taken.

In the final step, the in depth information collected in the previous steps is used to devise a therapeutic strategy to incorporate targeted therapies that will aid RT treatment by targeting the dominant and RT-resistant subpopulations, and potentially achieve long term tumor remission (FIG. 2F).

### Example 2: 10 Unbalanced Processes Give Rise to the Expression Variations of 11 Cell-Surface Proteins in 4T1 Mouse TNBC Cells

4T1 cells, obtained from a spontaneously developed tumor in an immunocompetent mouse model for stage IV TNBC, were irradiated using two doses (5 Gy or 15 Gy), and then grown under normal conditions for 24 h, 48 h and 6 days. The cells were then suspended and the expression levels of the selected panel of 11 cell-surface oncoproteins in single cells were measured using FACS. FIGS. 3A and 3B show the overall distributions of expression levels of the different proteins in the cells measured.

To gain insights regarding the behavior of the different proteins in single cells, initially 2D correlation plots were examined. For example, cMet and Her2 expression levels showed poor correlation in expression levels in response to RT (FIG. 3C). On the contrary, EGFR and Her2 levels demonstrated good correlation (FIG. 3D), as did MUC1 and cMet expression levels (FIG. 3E).

Note, however, that a good correlation between EGFR and Her2 does not necessarily suggest that they participate in the same unbalanced processes in all tested cells. Small subpopulations of cells unaffected by the same processes, and possibly displaying a poorer correlation between these proteins, may be overlooked when studying variations in all cells simultaneously (FIG. 3D, black circles). Similarly, small subpopulations of cells that demonstrate a good correlation between cMet and Her2 may exist, but nevertheless be masked by the representation shown in FIG. 3C. Moreover, the expression level of a certain protein can be influenced by several processes, due to non-linearity of biochemical processes: a certain pair of proteins can be correlated or non-correlated in the different unbalanced processes operating in the same cell, complicating even further the interpretation of these 2D correlation plots. Therefore, single cell SA was performed to map the unbalanced processes operating in the entire cellular population as well as in each single cell (see Methods for details).

The analysis revealed 10 unbalanced processes (i.e. altered protein-protein correlation patterns resulting from 10 constraints) which occurred in the untreated/treated cells (FIG. 3F, Table 3). The most abundant processes, indexed 1 and 2, appeared in 25% and 18% of the untreated cells, respectively. Processes 3 and 8, which included correlated Her2/EGFR and cMet/Muc1, correspondingly, initially demonstrated low abundancy, and appeared in 0.3% and 0.5% of the untreated cells, correspondingly (FIG. 3F, Table 2). Processes 3 and 8 became more dominant 6 days post-RT (Table 2; more details below).

TABLE 2
Calculating the percentages of cell subpopulation for
each single unbalanced process in 4T1 irradiated cells.

| | | 24 hrs. | 48 hrs. | 6 days |
|---|---|---|---|---|
| Process | Control | post IR | post IR | post IR |
| # 1 | 25% | 1.2% | 1.6% | 11.1% |
| # 2 | 18% | 14.6% | 10% | 11% |
| # 3 | 0.3% | 1.2% | 2.8% | 22% |
| # 4 | 0.4% | 0.3% | 0.2% | 0.5% |
| # 5 | 1.5% | 0.6% | 0.5% | 1.4% |
| # 6 | 0.2% | 0.02% | 0.08% | 0.2% |
| # 7 | 0.2% | 0.6% | 0.05% | 0.3% |
| # 8 | 0.5% | 0.7% | 1% | 4% |
| # 9 | 0.1% | 0.1% | 0.09% | 0.5% |
| # 10 | 0.1% | 0 | 0 | 0.06% |

TABLE 3
Genes active (upregulated/downregulated)
in each unbalanced process.

| | Proteins/Genes | Proteins/Genes |
|---|---|---|
| Process # | upregulated in the process | downregulated in the process |
| 1 | EGFR | Her2, CD44 |
| 2 | CD44 | EpCAM, MUC1, cMET |
| 3 | Her2, EGFR | N/A |
| 4 | CD24 | CD44 |
| 5 | CD44 | CD133 |
| 6 | PD-L1, E-cadherin | EpCAM |
| 7 | PD-L1, cKIT | Her2, CD44 |
| 8 | MUC1, cMET | E-cadherin |
| 9 | PD-L1 | cKIT |
| 10 | MUC1 | cMET |

The surprisal analysis revealed 10 unbalanced processes (i.e. altered protein-protein correlation patterns resulting from 10 constraints) which occurred in the untreated/treated cells. Five of the processes, (#1, #2, #3, #5 and #8) are appearing in at least 1% of the treated cells. The most abundant processes, indexed 1 and 2, appeared in 25% and 18% of the untreated cells, respectively. Processes 3 and 8, which included correlated Her2/EGFR and cMet/Muc1, correspondingly, initially demonstrated low abundancy, and appeared in 0.3% and 0.5% of the untreated cells, correspondingly. Processes 3 and 8 became more dominant 6 days post-RT.

### Example 3: 8 Sets of Unbalanced Processes, or 8 Distinct CSSS's, were Resolved, Suggesting that the Cells Form 8 Distinct Subpopulations

As mentioned above, more than one unbalanced process can operate in every cell. Therefore, to gain in depth information regarding the complete altered signaling signature in each cell, the sets of unbalanced processes in the cells studied, namely the CSSS, were inspected.

It was found that 8 different sets of unbalanced processes, representing 8 distinct signaling signatures (CSSS), repeated themselves in the population of cells before or after RT treatment (FIG. 3G). For the simplicity of representation, each CSSS was translated into a cell-specific barcode in which active/inactive processes are color-labeled (FIG. 3G). Note that only unbalanced processes with significant amplitudes were included in the CSSS of every individual cell (FIG. 3H-I, and Methods). FIG. 3J shows how selecting only cells with high amplitudes improve the correlation between the relevant proteins within the processes and thus the accuracy of the unbalanced processes in the analysis.

Additionally, only CSSS's that appeared in at least 1% of the cells were taken into account. The barcodes of these abundant subpopulations (each characterizing at least 1% of the cells) consisted of processes 1, 2, 3 and 8 (FIG. 3F-G).

Cells sharing the same set of unbalanced processes, or the same CSSS, were considered to be a subpopulation. These groups of cells all carry the same signaling signature, are influenced by the same altered processes, and are therefore expected to respond similarly to treatment.

### Example 4: The 8 Abundant Cellular Subpopulations Demonstrate Different Temporal Behaviors, and Different Variations in Abundance

Interestingly, when the temporal behavior of the abundant subpopulations was examined, they were found to be divided into 3 groups: (1) persistent subpopulations, which decreased 48 h post-RT and then returned to their previous size 6 d post-RT; (2) early subpopulations, which were minor initially (<1%), and then expanded 48 h and 6 d post-RT (>1%); (3) late subpopulations, which were small prior to RT and expanded only 6 d post-RT (FIG. 3G) A schematic representation of the different temporal behaviors is shown in FIG. 3K.

The persistent subpopulations did not change their abundance before RT and 6 days post-RT treatment. For example, subpopulation c comprised 14.5% of the cells before RT, and a similar percentage of the cells, 14.2%, was found to comprise this subpopulation 6 days post-RT. However, early and late subpopulations, b and f, respectively, expanded significantly 6 days post-RT.

Subpopulation b harbored only process 3 (FIGS. 3G and 3L), in which Her2, and to a lesser extent EGFR, were induced (FIGS. 3A, and 3M). Strikingly, subpopulation b was induced 60-fold post-irradiation relative to the non-irradiated cells (expanded from low (<1%) levels in untreated cells to ˜19-22% of the population, 6 days post-RT, FIG. 3N).

Subpopulation f harbored only process 8 (FIGS. 3G, and 3L), with induced cMet/MUC1 and reduced ECAD (FIG. 3A). Significant induction of subpopulation f was observed as well, from undetectable levels to ˜4% 6 days post-RT (FIG. 3N).

These results demonstrate an important concept: although cMet and Her2 were both induced in response to RT (FIG. 3A), CSSS-based analysis revealed that those two proteins were expressed in distinct cellular subpopulations (processes 3 and 8 do not appear in the same cells; FIG. 3G). Only some cells (<1%, participating in the less abundant subpopulations) shared both altered processes, 3 and 8 (not shown).

The development of such large, distinct and well-defined Her2+ and cMet+ subpopulations post-RT suggests that Her2 and cMet signaling may play a significant role in 4T1 cell survival and resistance in response to irradiation.

### Example 5: Simultaneous Inhibition of Her2 and cMet Sensitized 4T1 Cells to RT Treatment

It was hypothesized that simultaneous inhibition of both proteins, and thus targeting of both subpopulations, would sensitize 4T1 cells to RT treatment. Her2 and cMet represent good candidates for such a strategy, as they are both druggable oncoproteins, against which FDA-approved drugs exist.

To validate this hypothesis, each protein alone or in combination were inhibited, beginning 2 days prior to RT and until 6 days post-RT, and then cell survival was measured. Each drug alone (a Her2 inhibitor, Herceptin (H), or cMet inhibitor, Crizotinib (C)) was significantly less effective in sensitizing the cells to RT, relative to the combination of both targeted drugs (FIG. 3O). The combination of both drugs with RT induced higher killing rates of the cells and also brought about depletion of signaling downstream to Her2 and cMet, as indicated by the low levels of ERK1, Akt and S6K downstream signaling proteins and the enhanced cleavage of the apoptotic marker Casp3 (FIG. 3O-P).

### Example 6: Her2+ and cMet+ Cellular Subpopulations Expanded in Response to RT In Vivo

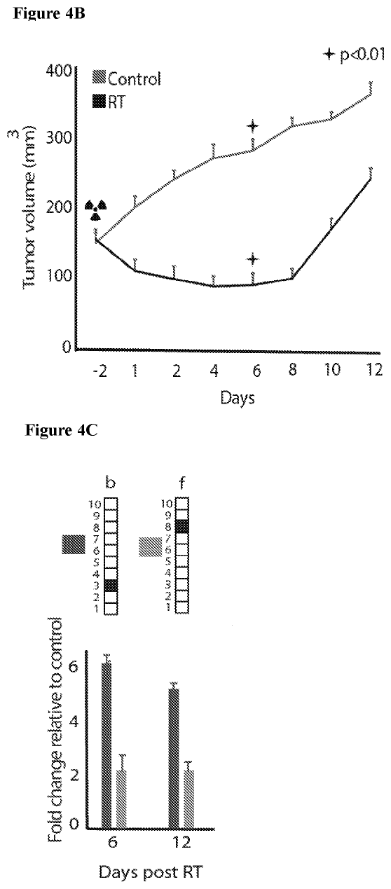

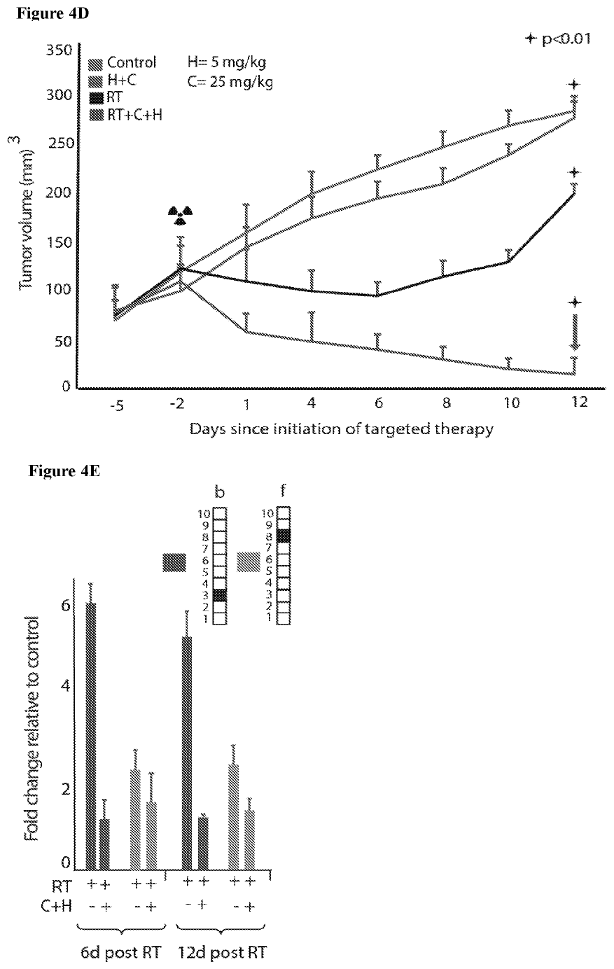

To validate the hypothesis further, 4T1 cells were implanted into Balb/c mice, an immunocompetent mouse model for TNBC. The cells were irradiated post-implantation using brachytherapy-focused irradiation technology adapted for mice²⁹ by CT imaging and Monte-Carlo based dosimetry (FIG. 4A). 4T1 tumors were then isolated and single cell suspensions were analyzed.

CSSS-based analysis of the tumors 6 days post-RT, when an initial shrinkage of tumors was observed (FIG. 4B), revealed an expansion of subpopulations b and f (FIG. 4C). Moreover, 12 days post-RT, when the tumors started growing again (FIG. 4B) the expanded subpopulations b and f were still detectable (FIG. 4C).

Inhibition of both Her2 and cMet proteins significantly sensitized the tumors to RT treatment (FIG. 4D). The combined treatment brought about shrinkage of the tumors and prevented development of resistance to RT (FIG. 4D, see the green arrow). The effect of RT plus the combined targeted therapy was highly synergistic in contrast to the effect of the two targeted drugs without RT, or RT treatment alone. Furthermore, the addition of the targeted drug combination (H+C) prior to RT brought about significant reduction in the size of subpopulations b and f (FIG. 4E). No other subpopulation expanded following treatment.

### Example 7: Targeting Her2+ and cMet+ Cellular Subpopulations Sensitized Human Cell Lines and Patient Derived TNBC Tumors to RT

To validate that the phenomenon of the expansion of Her2+ and cMet+ cellular subpopulations is not limited to TNBC mouse models, TNBC MDA-MB-231 and MDA-MB-468 human-derived cell lines, and TNBC patient-derived cells (BR45) were used.

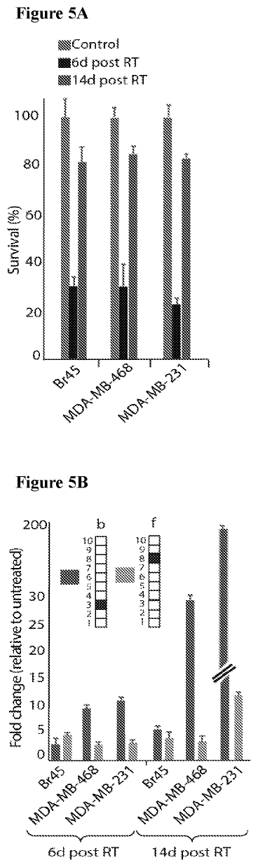

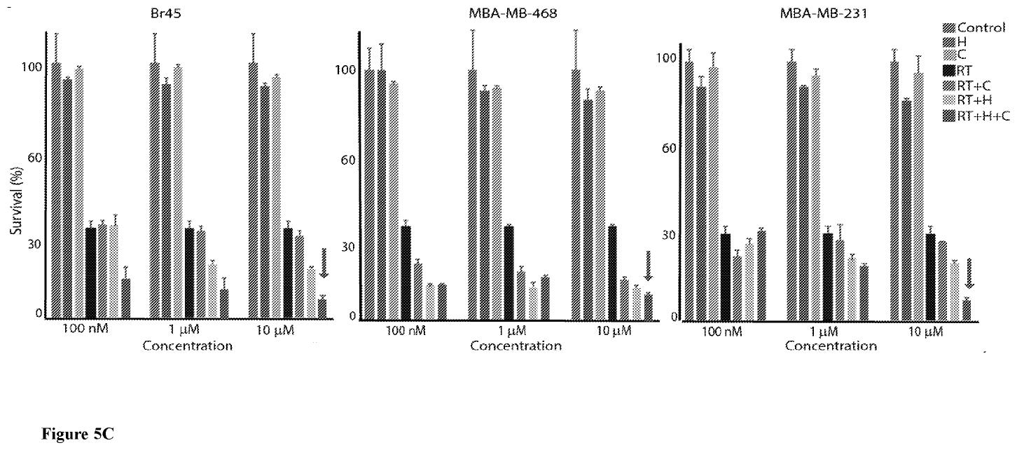

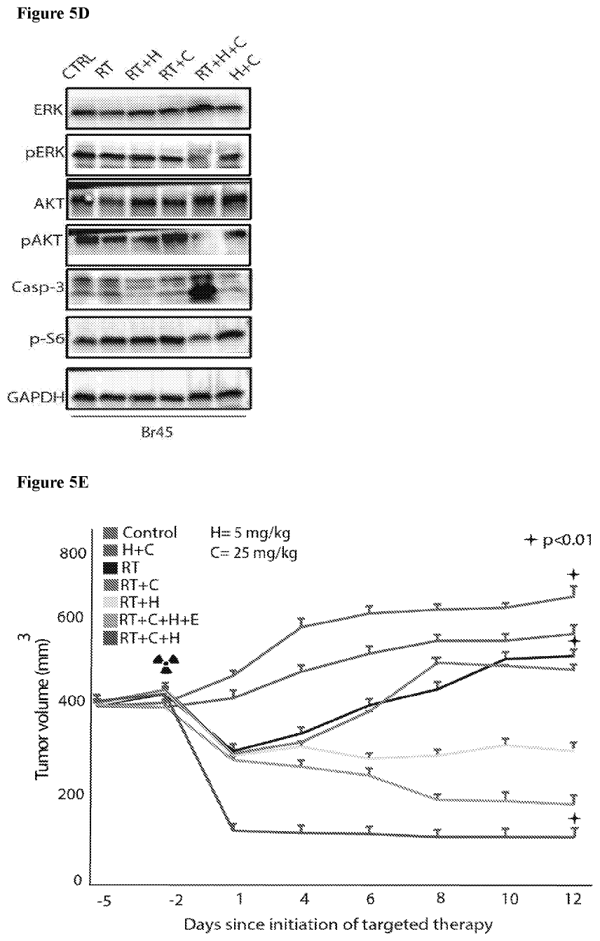

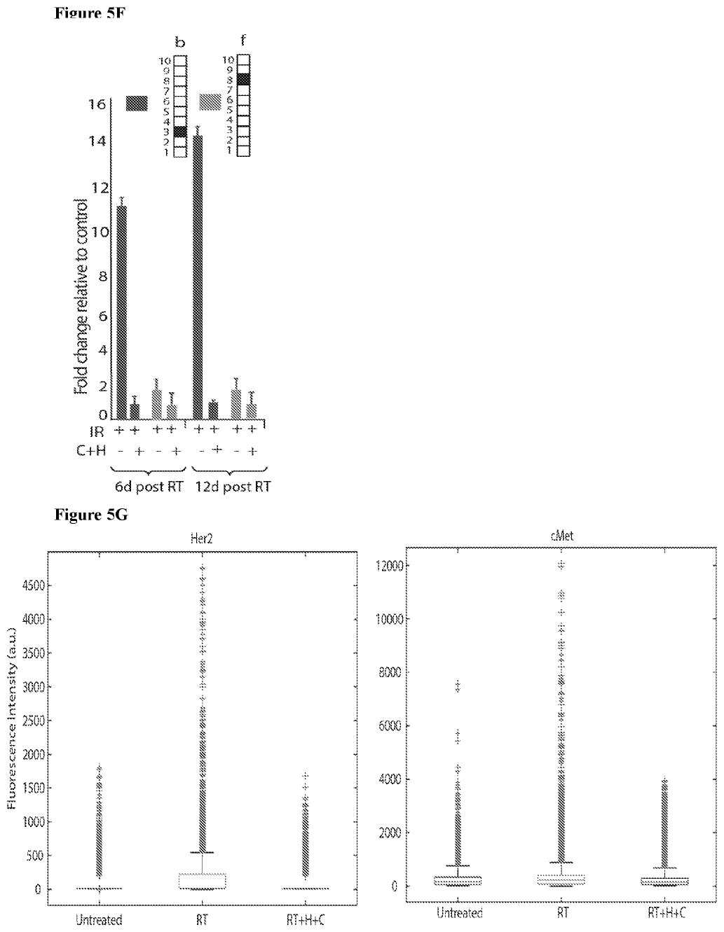

Inhibition of cell growth, observed in all cell types 6 days post-RT, was followed by significant regrowth of the cells 14 days post-RT (FIG. 5A). Subpopulations b and f, which expanded 6 days post-RT in all cell types, either kept their size or expanded even more following cellular regrowth, 14 days post-RT (FIG. 5B). Combined anti-Her2 and anti-cMet pretreatment sensitized all 3 types of human TNBC cells to RT (FIG. 5C). When combined with RT, each drug alone had a significantly smaller effect on cellular survival than the combination of both drugs together with RT (FIG. 5C, see green arrow). Moreover, depletion of the downstream pathways to Her2 and cMet signaling as well as induction of cleaved caspase 3 were observed when the cells were pretreated with anti-Her2 and anti-cMET inhibitors 1 day prior to RT (FIG. 5D).

Using patient-derived TNBC BR45 cells grown in PDX models, it was demonstrated that irradiated BR45 TNBC developed resistance to RT therapy in a short period of time (regrowth of the tumors was detected 7 days post-RT; FIG. 5E, see black curve). Pretreatment of the mice with each drug alone demonstrated a small effect on tumor growth (FIG. 5E). However, pretreatment of the mice with the combination of both drugs prior to RT brought about significant shrinkage of the tumor (FIG. 5E, dark green curve) and prevented resistance development.

Adding erlotinib (an EGFR inhibitor), which according to the algorithms was not expected to significantly influence tumor growth, indeed did not improve the results of the treatment (FIG. 5E). Subpopulations b and f were reduced when the targeted drug combination (H+C) was applied prior to RT (FIG. 5F-G). These results suggest that CSSS-based single cell resolution of the changes within TNBC tumor mass in response to RT provides guidance on how effective targeted drug combinations should be designed in order to sensitize tumors to RT and prevent development of resistance.

Importantly this approach allows for the mapping of distinct cellular subpopulations in one single tumor, which does not need to be compared to and analyzed with other tumors as is the case of bulk measurements. This single-cell resolution approach has numerous advantages over whole tumor, and multi-cancer analysis approaches.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of selecting a therapy for a cancer, the method comprising:
a. obtaining a sample comprising a population of cells derived from said cancer and subjecting said cells to a single-cell proteomic analysis;
b. receiving data from said single-cell proteomic analysis, wherein said data comprises a plurality of genes and/or proteins that deviate from a steady state;
c. calculating for said population of cells at least one unbalanced process active in at least one cell of said population of cells, wherein said calculating comprises performing a deterministic thermodynamic-based analysis on said data from said single-cell proteomic analysis, and assembling said genes and/or proteins deviating from a steady state into at least one unbalanced network(s)/signaling pathway(s), thereby producing a list of at least one calculated unbalanced process;
d. generating for a plurality of cells of said population of cells a cell-specific signature (CSS) comprising at least one unbalanced process from said list of at least one calculated unbalanced process active in each cell of said plurality of cells;
e. assigning all cells with the same CSS to a cellular subpopulation; and
f. selecting a therapy that targets said at least one unbalanced process in said CSS of at least one cellular subpopulation; thereby selecting a therapy.

2. The method of claim 1, wherein said receiving is receiving a single-cell proteomic analysis of a population of cells at a given timepoint.

3. The method of claim 1, wherein said sample is digested into a single-cell suspension before proteomic analysis of single cells.

4. The method of claim 1, wherein said proteomic analysis comprises single-cell FACS analysis.

5. (canceled)

6. The method of claim 1, wherein activity of said proteins, including oncogenic which is characteristic of tumors of said cancer.

7. (canceled)

8. The method of claim 1, wherein said cancer is a tumor of a subject, and said method is a method of selecting a subject-specific therapy.

9. (canceled)

10. The method of claim 1, wherein said deterministic thermodynamic-based analysis is a single-cell surprisal analysis.

11. The method of claim 1, wherein said calculating at least one unbalanced process active in at least one cell of said population of cells is calculating all unbalanced processes active in at least one cell of said population of cells based on said thermodynamic-based analysis.

12. The method of claim 1, wherein said CSS comprises all significantly unbalanced processes from said list of calculated unbalanced process active in cells of the population.

13. (canceled)

14. The method of claim 1, further comprising administering to a subject suffering from said cancer said selected therapy.

15. (canceled)

16. The method of claim 1, wherein said subpopulation of cells has been contacted by a first therapy prior to said proteomic analysis and said method is a method of selecting a second therapy, or a combination therapy of said first therapy and a second therapy.

17. (canceled)

18. (canceled)

19. (canceled)

20. The method of claim 16, further comprising receiving a single-cell proteomic analysis of a population of cells derived from said cancer before treatment with said first therapy, and assigning all cells of a plurality of cells of said population of cells derived from said cancer before treatment to a cellular population, wherein said selecting a second therapy is selecting a second therapy that targets a CSS of at least one cellular subpopulation that increased in abundance following said first therapy.

21. The method of claim 20, wherein said assigning comprises calculating for said population of cells derived before treatment at least two unbalanced processes active in at least one cell of said population of cells derived before treatment, wherein said calculating comprises performing a deterministic thermodynamic-based analysis on said proteomic analysis, thereby producing a list of calculated unbalanced processes; generating for said plurality of cells a CSS comprising all unbalanced processes from said list of calculated unbalanced processes active in each cell of said plurality of cells of said population of cells derived from said cancer before treatment, and assigning all cells with the same CSS to a cellular subpopulation.

22. The method of claim 20, comprising calculating the percent of all cells of said population before treatment that are in each cellular population, and calculating the percent of all cells of said population following treatment that are in each cellular population, and wherein an increase in abundance of a cellular subpopulation is an increase in the percent said subpopulation is of the total.

23. The method of claim 20, comprising selecting therapies that target CSS of a plurality of cellular subpopulations that increase in abundance following said first therapy.

24. (canceled)

25. The method of claim 16, wherein said first therapy is an untargeted/targeted cancer therapy.

26. (canceled)

27. (canceled)

28. (canceled)

29. (canceled)

30. (canceled)

31. (canceled)

32. (canceled)

33. The method of claim 1, further comprises assigning a barcode that indicates the active unbalanced processes in each of said CSS of said cancer.

34. (canceled)

35. (canceled)

36. (canceled)

37. (canceled)

38. (canceled)

39. (canceled)

40. (canceled)

41. The method of claim 1, wherein said therapy comprises s kit comprising an anti-Her2 therapy and an anti-cMet therapy and a label stating the anti-Her2 therapy and anti-cMet therapy are for use in combination with radiotherapy.

42. The kit of claim 41, for use in treating triple negative breast cancer in a subject in need thereof.

43. A computer program product for determining a therapy for a cancer, comprising a non-transitory computer-readable storage medium having program code embodied thereon, the program code executable by at least one hardware processor to
a. receive data from a single-cell proteomic analysis of a population of cells derived from a cancer, wherein said single-cell proteomic data comprises a plurality of genes and/or proteins that deviate from a steady state;
b. determine for said population of cells at least one unbalanced process active in at least one cell of said population of cells, wherein said determining comprises performing a deterministic thermodynamic-based analysis on said data from said single-cell proteomic analysis, and assembling genes and/or proteins deviating from a steady state into at least one unbalanced network(s)/signaling pathway(s), thereby producing a list of at least one determined unbalanced process;
c. generate for a plurality of cells of said population of cells a cell-specific signature (CSS) comprising at least one unbalanced process from said list of at least one determined unbalanced process active in each cell of said plurality of cells;
d. assign all cells with the same CSS to a cellular subpopulation; and
e. select and output a therapy that targets said at least one unbalanced process in said CSS of at least one cellular subpopulation.
